(19) United States
(12) Patent Application Publication
(10) Pub. No.: US 2026/0109995 A1
(43) Pub. Date: Apr. 23, 2026

(54) RSB11 SUPERIOR ALLELIC VARIANT RSB11-R AND APPLICATION THEREOF IN IMPROVING RICE SHEATH BLIGHT RESISTANCE

(71) Applicant: YANGZHOU UNIVERSITY, Yangzhou (CN)

(72) Inventors: Shimin ZUO, Yangzhou (CN); Zhiming FENG, Yangzhou (CN); Guangda WANG, Yangzhou (CN); Jianhua ZHAO, Yangzhou (CN); Peng GAO, Yangzhou (CN); Keming HU, Yangzhou (CN); Zongxiang CHEN, Yangzhou (CN); Yafang ZHANG, Yangzhou (CN); Wenya XIE, Yangzhou (CN)

(73) Assignee: YANGZHOU UNIVERSITY, Yangzhou (CN)

(21) Appl. No.: 19/121,837

(22) PCT Filed: Jul. 18, 2023
(86) PCT No.: PCT/CN2023/107807

§ 371 Date: Apr. 17, 2025

(30) Foreign Application Priority Data
Oct. 18, 2022 (CN) 202211271814.X

(51) Int. Cl. C12N 15/82; C07K 14/415; C12Q 1/6895

(52) U.S. Cl. CPC C12N 15/8282; C07K 14/415; C12Q 1/6895; C07K 2319/00; C12Q 2600/13; C12Q 2600/156; C12Q 2600/172

(57) ABSTRACT

Provided are an RSB11 superior allelic variant RSB11-R and an application thereof in improving rice sheath blight resistance. Also provided is a DNA molecule (an RSB11 superior allelic variant RSB11-R promoter), the nucleotide sequence thereof being as shown in SEQ ID NO: 4. The RSB11 superior allelic variant RSB11-R is transferred into the conventional japonica rice variety, and an improved rice plant with significantly enhanced sheath blight resistance can be obtained, which has important significance for genetic improvement of rice sheath blight resistance.

### CROSS REFERENCE TO THE RELATED APPLICATIONS

This application is the national phase entry of International Application No. PCT/CN2023/107807, filed on Jul. 18, 2023, which is based upon and claims priority to Chinese Patent Application No. 202211271814.X, filed on Oct. 18, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named KJ0106S_Sequence_Listing.xml, created on 04/16/2025, and is 32,109 bytes in size.

### TECHNICAL FIELD

The present invention relates to the field of biotechnology, specifically to the RSB11 superior allelic variant RSB11-R and its application in improving rice resistance to sheath blight.

### BACKGROUND

Sheath blight, caused by the pathogenic fungus Rhizoctonia solani Kühn, is one of the most devastating diseases affecting rice production in China. In recent years, cultivation practices such as straw returning, high-density planting, and drone-based pesticide application have led to the accumulation of pathogens in fields, exacerbating the incidence and severity of sheath blight. Annual yield losses caused by sheath blight range from 10% to 30%, reaching up to 50% in severe cases. Both the scale of infection and resultant yield loss caused by sheath blight consistently rank highest among all rice diseases, posing a significant threat to rice production security. Breeding disease-resistant rice varieties using resistance genes remains the most economical and effective strategy for disease control. Different rice varieties have obvious differences in resistance to sheath blight. However, no rice variety or germplasm that is completely resistant to sheath blight has been found so far, and resistant varieties are also seriously lacking.

Rice resistance to sheath blight is a typical quantitative trait controlled by quantitative trait loci (QTLs) or polygenes. Over 60 QTLs associated with sheath blight resistance have been identified. Nevertheless, it is difficult to accurately identify the phenotypes of different individuals in a genetically isolated population, there has been no report of successful cloning of QTLs for resistance to sheath blight using traditional positional cloning methods, and only a few QTLs have been proven to have breeding application value, which seriously restricts the analysis of the molecular mechanism of resistance to sheath blight and the breeding process. In addition, through reverse genetics and various omics strategies, it was found that many genes or signaling pathways in the known plant defense system are involved in the regulation of sheath blight resistance, such as genes related hormones (salicylic acid, jasmone and ethylene), coding gene of pathogenesis-related proteins, sugar transporters, transcription factors and chlorophyll degradation proteins, etc. These advances have greatly deepened our understanding of the interaction mechanisms between rice and sheath blight pathogens. However, most of these genes usually require precise regulation during growth and development. When they are overactivated or continuously inhibited, although disease resistance is enhanced, growth and development are often affected. Therefore, their application value in breeding needs further study.

In recent years, with the development of high-throughput genotyping technology, genome-wide association study (GWAS) based on linkage disequilibrium (LD) has shown great advantages in aspect of mining QTL/genes. Compared with traditional map-based cloning methods, GWAS can identify single nucleotide polymorphism (SNP) marker sites that are closer to candidate genes and mine favorable alleles of target traits in natural varieties. For sheath blight resistance, Chen et al. used 299 different rice varieties to identify 11 SNP sites significantly associated with sheath blight resistance through GWAS. Zhang et al. used 563 rice varieties in the 3K Rice Genome Project and detected 27 sites significantly associated with sheath blight resistance through GWAS. Li et al. cloned an excellent allele ZmFBL41B73 that confers resistance to sheath blight in maize through GWAS and found that the gene mainly enhances resistance by increasing the lignin content in the cell wall, a mechanism that is also conducive to enhancing rice resistance to sheath blight. Recently, Wang et al. conducted a GWAS study on 259 different rice varieties and proved that two genes, OsRSR1 and OsRLCK5, improve sheath blight resistance by regulating ROS balance. These studies show that the application of GWAS is expected to greatly accelerate the identification of excellent alleles for sheath blight resistance in natural rice varieties and the research process of disease resistance mechanism. However, the application value of these cloned sheath blight resistance genes has not been confirmed in breeding practice and is far from meeting the needs of breeding.

Overall, the available sheath blight resistance gene resources are very scarce in rice disease resistance breeding. Therefore, further mining and cloning of quantitative sheath blight resistance genes with breeding utilization value will provide important gene resources for molecular breeding of rice sheath blight resistance.

### SUMMARY

The purpose of the present invention is to provide RSB11 superior allele RSB11-R and use in improving rice sheath blight resistance thereof.

In the first aspect, the present invention claims a DNA molecule, which is a promoter of RSB11 superior allele RSB11-R, referred to as RSB11-R promoter.

The nucleotide sequence of the DNA molecule claimed by the present invention is shown in SEQ ID NO: 4.

In the second aspect, the present invention claims a recombinant vector, expression cassette, transgenic cell line or recombinant bacteria containing the DNA molecule described in the first aspect above.

In the third aspect, the present invention claims the use of the DNA molecule described in the first aspect above as a promoter in enhancing the expression of a target gene in a plant.

Furthermore, the target gene is a nucleic acid molecule capable of expressing RSB11 protein.

The RSB11 protein is anyone of the following:
(A1) a protein having an amino acid sequence of SEQ ID NO: 1;
(A2) a protein derived from rice with the same function of SEQ ID NO: 1 and have the amino acid sequence obtained by replacing and/or deleting and/or adding one or more amino acid residues of the amino acid sequence shown in SEQ ID NO: 1;
(A3) a protein having more than 99%, more than 95%, more than 90%, more than 85% or more than 80% identity with the amino acid sequence defined in any one of (A1)-(A2) and derived from rice with the same function of the amino acid sequence defined in any one of (A1)-(A2);
(A4) a fusion protein obtained by connecting a protein tag to the N-terminus and/or C-terminus of protein of anyone of (A1)-(A3).

In the above proteins, the protein tag refers to a polypeptide or protein fused and expressed with the target protein using DNA in vitro recombination technology to facilitate the expression, detection, tracing and/or purification of the target protein. The protein tag can be a Flag tag, a His tag, an MBP tag, an HA tag, a myc tag, a GST tag and/or a SUMO tag, etc.

In the above proteins, identity refers to the identity of the amino acid sequence. The identity of the amino acid sequence can be determined using homology search sites on the Internet, such as the BLAST page on the NCBI homepage website. For example, in Advanced BLAST2.1, by using blastp as the program, setting the Expect value to 10, setting all filters to OFF, using BLOSUM62 as the Matrix, setting the Gap existence cost, Per residue gap cost and Lambda ratio to 11, 1 and 0.85 (default values) respectively, and searching for the identity of a pair of amino acid sequences, the identity value (%) can be obtained.

In the above proteins, the identity of more than 95% can be at least 96%, 97%, 98% identity. The identity of more than 90% can be at least 91%, 92%, 93%, 94% identity. The identity of more than 85% can be at least 86%, 87%, 88%, 89% identity. The above 80% identity may be at least 81%, 82%, 83%, 84% identity.

In the fourth aspect, the present invention claims the use of the DNA molecule of the first aspect in anyone of the following (a1)-(a2):
(a1) improving plant resistance to sheath blight;
(a2) improving plant resistance to Rhizoctonia solani Kühn.

At the same time, plant yield loss can also be reduced.

In the use, the DNA molecule initiates the expression of a target gene in the plant, and the target gene is a nucleic acid molecule capable of expressing RSB11 protein.

Wherein, the RSB11 protein is the protein shown in anyone of (A1)-(A4) above.

In the fifth aspect, the present invention claims a primer pair.

The primer pair claimed by the present invention consists of two single-stranded DNA molecules shown in SEQ ID NO: 5 and SEQ ID NO: 6.

The primer pair is used to amplify a fragment of an SNP site related to sheath blight resistance contained in the promoter region of the RSB11 gene in the rice genome.

In the sixth aspect, the present invention claims a kit containing the primer pair described in the fifth aspect.

The kit claimed in the present invention also contains restriction endonuclease MluI.

In the seventh aspect, the present invention claims the use of the DNA molecule described in the first aspect above, the primer pair described in the fifth aspect above, or the kit described in the sixth aspect above in plant breeding.

In the eighth aspect, the present invention claims protection for anyone of the following uses:
M5: use of a substance for detecting the polymorphism or genotype of the four variant sites SNP94782, Indel1171, Indel946 and SNP94780 in identifying or assisting in identifying rice resistance to sheath blight; the SNP94782 is an SNP in the rice genome, corresponding to the 516th nucleotide of SEQ ID NO: 4 (RSB11-R promoter sequence), which is T or G; Indel1171 is a deletion variation in the rice genome, corresponding to the 2005-2135th nucleotides (131 bp) of SEQ ID NO: 4 (RSB11-R promoter sequence), which is deletion or non-deletion; Indel946 is a deletion variation in the rice genome, corresponding to the nucleotides 2231-2486 (256 bp) of SEQ ID NO: 4 (RSB11-R promoter sequence), which is deletion or non-deletion; the SNP94780 is an SNP in the rice genome, corresponding to the nucleotide 1653 of SEQ ID NO: 2 or SEQ ID NO: 3 (RSB11 gene sequence), which is A or G.
M6: Use of a substance for detecting haplotypes in identifying or assisting in identifying rice resistance to sheath blight; the haplotype is a polymorphism or genotype combination of the four variable sites SNP94782, Indel1171, Indel946 and SNP94780 in M5 on the rice genome;
M7: use of a substance for detecting the polymorphism or genotype of SNP94782 in M5 in identifying or assisting in identifying rice resistance to sheath blight;
M8: use of the primer pair described in the fifth aspect or the kit described in the sixth aspect in detecting the polymorphism or genotype of SNP94782 in M5;
M9: use of the primer pair described in the fifth aspect or the kit described in the sixth aspect in identifying or assisting in identifying rice resistance to sheath blight.

In each of the above uses, the plant may be a monocot or a dicot.

Further, the monocot may be a grass.

Further, the grass may be a rice plant.

More specifically, the rice plant may be rice.

In the ninth aspect, the present invention claims anyone of the following methods:
Q5: A method for identifying or assisting in identifying rice resistance to sheath blight, including the following steps (C1) or (C2):
(C1) detecting the haplotype described in M6 in the eighth aspect above in the genome of the rice to be tested, and determining the resistance of the rice to be tested to sheath blight according to the haplotype of the rice to be tested as follows: The resistance of the homozygous genotype rice corresponding to the haplotype RSB11-R to sheath blight is stronger or is a candidate for being stronger than the homozygous gene corresponding to the haplotype RSB11-S type rice; the haplotype RSB11-R is: the SNP94782 is T, the Indel1171 is not deletion, the Indel946 is non-deletion, and the SNP94780 is A; the haplotype RSB11-S is: the SNP94782 is G, the Indel1171 is deletion, the Indel946 is deletion, and the SNP94780 is G;
furthermore, the method for detecting the haplotype in the rice genome to be tested can be sequencing.
(C2) Detecting the SNP94782 in M5 of the eighth aspect above in the genome of the rice to be tested, and determining the resistance of the rice to be tested to sheath blight according to the genotype of the SNP94782 of the rice to be tested as follows: the resistance of the rice with the genotype of SNP94782 being TT to sheath blight is stronger than or is a candidate to be stronger than the resistance of the rice with the genotype of SNP94782 being GG to sheath blight;
Q6: a method for cultivating a rice variety with improved resistance to sheath blight, including the following steps: a rice variety identified by the method described in Q5 with relatively strong resistance to sheath blight (the haplotype is haplotype RSB11-R, or the genotype of SNP94782 is TT) is selected as the donor parent, and a rice variety identified by the method described in Q5 with relatively weak resistance to sheath blight but with expected agronomic traits (such as the haplotype is haplotype RSB11-S, or the genotype of SNP94782 is GG) is selected as the recurrent parent, and a rice variety with improved resistance to sheath blight and the expected agronomic traits is obtained through continuous backcrossing.

Further, in step (C2) of the method described in Q5, the primer pair described in the fifth aspect or the kit described in the sixth aspect are used to detect the genotype of SNP94782 in the rice genome to be tested.

Furthermore, the rice genome DNA to be tested is used as a template, and the primer pair is used for amplification. If a target fragment of 154 bp is obtained, as shown in SEQ ID NO: 7, and the 28th position is homozygous T, the genotype of SNP94782 in the rice genome to be tested is TT; if a target fragment of 154 bp is obtained, as shown in SEQ ID NO: 7, and the 28th position is homozygous G, the genotype of SNP94782 in the rice genome to be tested is GG.

Further, the rice genome DNA to be tested is used as a template, and the primer pair is used for amplification, and the amplified product is completely digested with MluI. If the digestion product is 154 bp, the genotype of SNP94782 in the rice genome to be tested is TT; if the digestion products are 130 bp and 24 bp, the genotype of SNP94782 in the rice genome to be tested is GG.

In the tenth aspect, the present invention claims to protect the use of RSB11 protein or related biological materials thereof in anyone of the following:
P1, regulating plant resistance to sheath blight;
P2, regulating plant resistance to Rhizoctonia solani Kühn.

The RSB11 protein may be anyone of the proteins shown in (A1)-(A4) above.

The related biological material is a nucleic acid molecule capable of expressing the RSB11 protein, or an expression cassette, a recombinant vector, a recombinant microorganism or a transgenic cell line containing the nucleic acid molecule.

The expression cassette refers to a DNA capable of expressing RSB11 in a host cell, which may include not only a promoter for initiating transcription of the RSB11 gene, but also a terminator for terminating RSB11 transcription. Furthermore, the expression cassette may also include an enhancer sequence. Promoters that can be used in the present invention include, but are not limited to, constitutive promoters, tissue, organ and development-specific promoters, and inducible promoters. Examples of promoters include, but are not limited to, the Ubiqutin promoter (pUbi); the constitutive promoter 35S of cauliflower mosaic virus; the wound-inducible promoter from tomato, leucine aminopeptidase (“LAP”, Chao et al. (1999) Plant Physiol 120:979-992); the chemically inducible promoter from tobacco, pathogenesis-related 1 (PRI) (induced by salicylic acid and BTH (benzothiadiazole-7-thiocarboxylic acid S-methyl ester)); the tomato proteinase inhibitor II promoter (PIN2) or the LAP promoter (both of which can be induced by methyl jasmonate); heat shock promoters (U.S. Pat. No. 5,187,267); tetracycline-inducible promoters (U.S. Pat. No. 5,057,422); seed-specific promoters, such as millet seed-specific promoter pF128 (CN101063139B (China Patent No. 2007 1 0099169.7)), seed storage protein-specific promoters (e.g., promoters of phaseolin, napin, oleosin, and soybean beta conglycin (Beachy et al. (1985) EMBO J. 4:3047-3053)). They can be used alone or in combination with other plant promoters. All references cited herein are cited in their entirety. Suitable transcription terminators include, but are not limited to, the Agrobacterium nopaline synthase terminator (NOS terminator), the cauliflower mosaic virus CaMV 35S terminator, the tml terminator, the pea rbcS E9 terminator, and the nopaline and octopine synthase terminators (see, e.g., Odell et al. (1985) Nature 313:810; Rosenberg et al. (1987) Gene, 56:125; Guerineau et al. (1991) Mol. Gen. Genet, 262:141; Proudfoot (1991) Cell, 64:671; Sanfacon et al. Genes Dev., 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627).

Construct a recombinant expression vector containing the RSB11 gene expression cassette. The plant expression vector used can be a binary Agrobacterium vector or a Gateway system vector, such as pBin438, pCAMBIA1302, pCAMBIA2301, pCAMBIA1301, pCAMBIA1300, pBI121, pGWB411, pGWB412, pGWB405, pCAMBIA1391-Xa or pCAMBIA1391-Xb. When RSB11 is used to construct a recombinant expression vector, any enhanced, constitutive, tissue-specific or inducible promoter can be added before its transcription start nucleotide, such as cauliflower mosaic virus (CAMV) 35S promoter, ubiqutin promoter (pUbi), etc., which can be used alone or in combination with other plant promoters; in addition, when the gene of the present invention is used to construct a plant expression vector, enhancers can also be used, including translation enhancers or transcription enhancers. These enhancer regions can be ATG start codons or adjacent region start codons, etc., but must be the same as the reading frame of the coding sequence to ensure the correct translation of the entire sequence. The sources of the translation control signal and the start codon are extensive and can be natural or synthetic. The translation start region can come from the transcription start region or the structural gene.

In order to facilitate the identification and screening of transgenic plant cells or plants, the plant expression vector used can be processed, such as adding genes that can be expressed in plants and encode enzymes or luminescent compounds that can produce color changes (GUS genes, luciferase genes, etc.), antibiotic markers with resistance (gentamicin markers, kanamycin markers, etc.) or chemical reagent resistance marker genes (such as herbicide resistance genes), etc.

In the above uses, the vector can be a plasmid, clay, phage or virus vector.

In the above uses, the microorganism can be yeast, bacteria, algae or fungi. The bacteria can be from Escherichia, Erwinia, Agrobacterium (such as Agrobacterium tumefaciens EHA105), Flavobacterium, Alcaligenes, Pseudomonas, Bacillus, etc.

In the plant, the expression amount and/or activity of the RSB11 protein increases, and the resistance to sheath blight and/or resistance to Rhizoctonia solani Kühn increases;

In the plant, the expression amount and/or activity of the RSB11 protein decreases, and the resistance to sheath blight increases and/or resistance to Rhizoctonia solani Kühn decreases.

Wherein, the nucleic acid molecule capable of expressing the RSB11 protein may be DNA, such as cDNA, genomic DNA or recombinant DNA; the nucleic acid molecule may also be RNA, such as mRNA, etc.

Furthermore, the nucleic acid molecule capable of expressing the RSB11 protein may be anyone of the following:
(B1) a DNA molecule shown in SEQ ID NO: 2 or SEQ ID NO: 3;
(B2) a DNA molecule that hybridizes with the DNA molecule defined in (B1) under stringent conditions and encodes the RSB11 protein;
(B3) a DNA molecule that has a homology of more than 99%, more than 95%, more than 90%, more than 85% or more than 80% with anyone of the DNA sequences defined in (B1)-(B2) and encodes the RSB11 protein.

In the above nucleic acid molecules, the stringent conditions may be as follows: 50° C., hybridization in a mixed solution of 7% sodium dodecyl sulfate (SDS), 0.5M Na3PO4 and 1 mM EDTA, rinsed at 50° C., 2×SSC, 0.1% SDS; it may also be: 50° C., hybridization in a mixed solution of 7% SDS, 0.5M Na3PO4 and 1 mM EDTA, rinsed at 50° C., 1×SSC, 0.1% SDS; it may also be: 50° C., hybridization in a mixed solution of 7% SDS, 0.5M Na3PO4 and 1 mM EDTA, rinsed at 50° C., 0.5×SSC, 0.1% SDS; it may also be: 50° C., hybridization in a mixed solution of 7% SDS, 0.5M Na3PO4 and 1 mM EDTA, rinsed at 50° C., 0.1×SSC, 0.1% SDS; it may also be: 50° C., hybridization in a mixed solution of 7% SDS, 0.5M Na3PO4 and 1 mM EDTA, rinsed at 50° C., 0.1×SSC, 0.1% SDS; it may also be: 50° C., 7% Hybridize in a mixed solution of SDS, 0.5M Na3PO4 and 1 mM EDTA, rinse in 0.1×SSC, 0.1% SDS at 65° C.; or: hybridize in a solution of 6×SSC, 0.5% SDS at 65° C., and then wash the membrane once with 2×SSC, 0.1% SDS and 1×SSC, 0.1% SDS.

In the above nucleic acid molecules, homology refers to the identity of the nucleotide sequence. The identity of the nucleotide sequence can be determined using homology search sites on the Internet, such as the BLAST page on the NCBI homepage website. For example, in Advanced BLAST2.1, by using blastp as the program, setting the Expect value to 10, setting all filters to OFF, using BLOSUM62 as the Matrix, setting the Gap existence cost, Per residue gap cost and Lambda ratio to 11, 1 and 0.85 (default value) respectively, and searching for the identity of a pair of nucleotide sequences, the identity is calculated, and then the identity value (%) can be obtained.

In the above nucleic acid molecules, the homology of more than 95% may be at least 96%, 97%, 98% identity. The homology of more than 90% may be at least 91%, 92%, 93%, 94% identity. The homology of more than 85% may be at least 86%, 87%, 88%, 89% identity. The homology of more than 80% may be at least 81%, 82%, 83%, 84% identity.

In the eleventh aspect, the present invention claims anyone of the following uses:
M3: use of a substance capable of increasing the expression level and/or activity of RSB11 protein in plants in anyone of the following (a1)-(a2);
(a1) improving plant resistance to sheath blight;
(a2) improving plant resistance to Rhizoctonia solani Kühn;

At the same time, it can also reduce plant yield losses.
M4: Use of a substance capable of reducing the expression level and/or activity of RSB11 protein in a plant in anyone of the following (b1)-(b2):
(b1) reducing the resistance of a plant to sheath blight;
(b2) reducing the resistance of a plant to Rhizoctonia solani Kühn.

Wherein, the RSB11 protein may be anyone of the proteins shown in (A1)-(A4) above.

Wherein, the plant may be a monocot or a dicot.

Further, the monocot may be a grass plant.

Further, the grass plant may be a rice plant.

More specifically, the rice plant may be rice.

In the twelfth aspect, the present invention claims anyone of the following methods:
Q1: a method for cultivating plants with enhanced resistance to sheath blight and/or enhanced resistance to Rhizoctonia solani Kühn, including the step of increasing the expression level and/or activity of RSB11 protein in a recipient plant.

The method can be achieved by hybridization or transgenic means. The method can also reduce plant yield losses.
Q2: A method for cultivating plants with reduced resistance to sheath blight and/or reduced resistance to Rhizoctonia solani Kühn, including the step of reducing the expression level and/or activity of RSB11 protein in a recipient plant.

The method can be achieved by hybridization or transgenic means.
Q3: A method for cultivating transgenic plants with enhanced resistance to sheath blight and/or enhanced resistance to Rhizoctonia solani Kühn, including the following steps: introducing a nucleic acid molecule capable of expressing RSB11 protein into a recipient plant to obtain a transgenic plant; the transgenic plant has enhanced resistance to sheath blight and/or enhanced resistance to Rhizoctonia solani Kühn compared with the recipient plant.

In the method, the introduction of a nucleic acid molecule capable of expressing the RSB11 protein into the recipient plant can be achieved by any technical means capable of achieving this purpose. For example, the recombinant vector described in the first aspect above is introduced into the target plant.

In an embodiment of the present invention, the recombinant vector is specifically a recombinant plasmid obtained by cloning a nucleic acid molecule capable of expressing the RSB11 protein into a pCAMBIA2300 vector. The promoter that initiates the transcription of the nucleic acid molecule capable of expressing the RSB11 protein in the recombinant plasmid is the RSB11-R promoter (i.e., the DNA molecule shown in SEQ ID NO: 4) (corresponding to the complementary vector in the embodiment) or the Ubi promoter (corresponding to the overexpression vector in the embodiment).

The method can simultaneously reduce plant yield loss.
Q4: A method for cultivating transgenic plants with reduced resistance to sheath blight and/or reduced resistance to Rhizoctonia solani Kühn, including the following steps: inhibiting the expression of a nucleic acid molecule capable of expressing the RSB11 protein in a recipient plant to obtain a transgenic plant; the transgenic plant has reduced resistance to sheath blight and/or reduced resistance to Rhizoctonia solani Kühn compared to the recipient plant.

In the method, inhibiting the expression of the nucleic acid molecule capable of expressing the RSB11 protein in the recipient plant can be achieved by any technical means that can achieve this purpose.

In an embodiment of the present invention, it is specifically achieved by CRISPR/Cas9 technology. Further, the specific spacer sequence targeting the RSB11 gene is ATACCCTCGCGGTGGGGC (SEQ ID NO: 8).

The RSB11 protein may be anyone of the proteins shown in (A1)-(A4) above.

In the above method, the recombinant vector is introduced into the recipient plant, specifically by using conventional biological methods such as Ti plasmid, Ri plasmid, plant virus vector, direct DNA transformation, microinjection, electroporation, agrobacterium-mediated transformation, etc. to transform plant cells or tissues, and cultivate the transformed plant tissues into plants.

In the above method, the transgenic plant is understood to include not only first-generation to second-generation transgenic plants, but also their progeny. For transgenic plants, the gene can be propagated in the species, and the gene can also be transferred into other varieties of the same species using conventional breeding techniques, especially including commercial varieties. The transgenic plants include seeds, callus, complete plants and cells.

In the method, the nucleic acid molecule capable of expressing the RSB11 protein can be DNA, such as cDNA, genomic DNA or recombinant DNA; the nucleic acid molecule can also be RNA, such as mRNA, etc.

Further, the nucleic acid molecule capable of expressing the RSB11 protein may be anyone of the following:
(B1) a DNA molecule shown in SEQ ID NO: 2 or SEQ ID NO: 3;
(B2) a DNA molecule that hybridizes with the DNA molecule defined in (B1) under stringent conditions and encodes the RSB11 protein;
(B3) a DNA molecule that has more than 99%, more than 95%, more than 90%, more than 85% or more than 80% homology with anyone of the DNA sequences defined in (B1)-(B2) and encodes the RSB11 protein.

Wherein, the plant may be a monocot or a dicot.

Further, the monocot may be a grass plant.

Further, the grass plant may be a rice plant.

More specifically, the rice plant may be rice.

### BRIEF DESCRIPTION OF THE DRAWINGS

FIGS. 1A-1G show the RSB11 gene significantly associated with rice sheath blight resistance identified by genome-wide association analysis. FIG. 1A: Manhattan plot of GWAS analysis of sheath blight resistance, arrows indicate the two most significantly associated SNP sites. FIG. 1B: Local Manhattan plot of the LD interval of the two most significantly associated SNP sites and the genes within the interval. FIG. 1C: Expression of 24 genes in the LD interval induced by sheath blight in Xiangwanxian No. 7 variety. FIG. 1D: Association analysis results based on RSB11 sequence variation, dots indicate SNPs, and triangles indicate Indels. FIG. 1E: The RSB11 gene is divided into two haplotypes based on the four most significantly associated sites. n indicates the number of varieties in each haplotype. The bar graph indicates the sheath blight disease level of each haplotype group. FIG. 1F: Comparison of RSB11 expression levels before and after sheath blight infection in RSB11-R and RSB11-S varieties. FIG. 1G: Transient expression assay of rice protoplast promoter activity. pRSB11-S and pRSB11-R represent the promoter region of RSB11 in Zhendao 88 and Xiangwanxian 7 varieties, respectively. P indicates the significance of the two-sided t test.

FIGS. 2A-2E show that the RSB11 T-DNA insertion mutant rsb11 is more susceptible to sheath blight. FIG. 2A: T-DNA insertion site in rsb11. P1, P2 and P3 represent primers used to verify the insertion site. RB and LB represent the right and left borders of T-DNA, respectively. FIG. 2B: The RSB11 gene in rsb11 is destroyed and not expressed (electrophoresis). FIG. 2C: PCR verification of the insertion site. FIG. 2D and FIG. 2E: Comparison of sheath blight resistance in the field between rsb11 and WT.

FIGS. 3A-3F show the transgenic verification of RSB11 regulating sheath blight resistance. FIG. 3A: Expression level of RSB11 in RSB11 complementation line. FIG. 3B: Expression level of RSB11 in RSB11 overexpression line. FIG. 3C: Sheath blight resistance of RSB11 complementation line. FIG. 3D: Sheath blight resistance of RSB11 overexpression line. FIG. 3E: Mutation site of RSB11 knockout line, where the sequence of WT is shown in SEQ ID NO: 22, the sequence of rsb11-ko1 is shown in SEQ ID NO: 23, the sequence of rsb11-ko2 is shown in SEQ ID NO: 24, the sequence of rsb11-ko3 is shown in SEQ ID NO: 25. FIG. 3F: Sheath blight resistance of RSB11 knockout line. Different capital letters indicate multiple comparison results at the P<0.01 level. ** indicates significant level P<0.01.

FIGS. 4A-4D show the field sheath blight resistance and main agronomic traits of RSB11 complementation, overexpression and knockout lines. Different capital letters indicate multiple comparison results at the P<0.01 level. FIG. 4A: Field sheath blight resistance phenotype of RSB11 complementation line and wild type (WT). FIG. 4B: Field sheath blight resistance phenotype of RSB11 overexpression, knockout and wild type. FIG. 4C: Comparison of main agronomic traits of RSB11 complementation line and wild type (WT). FIG. 4D: Comparison of main agronomic traits of RSB11 overexpression, knockout and wild type. N.S. indicates no significant difference.

FIGS. 5A-5B show a marker-assisted selection route for the superior allele RSB11-R. FIG. 5A: RSB11-R specific dCAPs molecular marker dCAPs-2697. FIG. 5B: MAS route for introducing RSB11-R in YSBR1 into japonica rice variety TG394.

FIGS. 6A-6L show that RSB11-R significantly reduced the yield loss caused by sheath blight. FIG. 6A: Sheath blight resistance of NIL-RSB11-R and TG394. FIG. 6B: Sheath blight phenotypes of NIL-RSB11-R and TG394 under severe disease conditions in the field. FIGS. 6C-6L: Disease level (FIG. 6C), plot yield (FIG. 6D), fruiting rate (FIG. 6E), 1000-grain weight (FIG. 6F), number of effective panicles (FIG. 6G), number of grains per panicle (FIG. 6H), plant height (FIG. 6I), full growth period (FIG. 6J), chalky grain rate (FIG. 6K) and amylose content (FIG. 6L) of NIL-RSB11-R and TG394 under mild and severe disease conditions. Different uppercase and lowercase letters indicate the results of multiple comparisons at the P<0.01 and P<0.05 levels, respectively. ** indicates a significant level of P<0.01. N.S. indicates no significant difference.

### DETAILED DESCRIPTION OF THE EMBODIMENTS

The following examples are provided for a better understanding of the present invention, but are not intended to limit the present invention. The experimental methods in the following examples are conventional methods unless otherwise indicated. The test materials used in the following examples are purchased from conventional biochemical reagent stores unless otherwise indicated. The quantitative tests in the following examples were repeated three times, and the results were averaged.

### Example 1: Identification, Cloning and Functional Verification of RSB11 Gene

### I. Material and Method

### (1) Identification of Resistance to Sheath Blight

Identification of resistance to sheath blight was carried out according to the method described by previous researchers (He Min et al., Acta Botanica Sinica, 2020, 55: 577-587). The highly pathogenic sheath blight fungus strain RH-9 (Zuo et al., Theoretical and Applied Genetics, 2013, 126: 1257-1272) was used to inoculate rice. Sheath blight fungi were first cultured on potato dextrose agar medium at 28° C. for 3 days. Then, fungal blocks (about 0.7 cm in diameter) were transferred to potato dextrose broth medium containing wood bark with a thickness of 0.8 mm and a length of 1.0 cm and grown at 28° C. for about 3 days until the mycelium completely covered the wood bark. The wood bark colonized with mycelium was used as the inoculum. For field inoculation, the inoculum was inserted from the top of the plant into the inner side of the third sheath at the late tillering stage, and three main tillers were inoculated on each plant. According to the “0-9” disease scoring system (Zuo et al., Theoretical and Applied Genetics, 2013, 126:1257-1272), the disease level was recorded 30 days after heading, with three replicates, 10 plants in each replicate, and the average disease level of the three replicates was finally calculated. For greenhouse inoculation, the plants were transferred to a greenhouse with a relative humidity of 75%-85% in the early stage of heading, and the four main tillers of each plant were inoculated in the same way as field inoculation. The length of the lesions was measured 14 days after inoculation, and the average of the three replicates was calculated.

### (2) Fluorescence Quantitative PCR

Total RNA was extracted from rice leaf sheaths using TRizol reagent (Invitrogen, Carlsbad, CA). According to the instructions of the reagent (PrimeScript™ 1st strand cDNA Synthesis Kit, TaKaRa), 1 μg of purified total RNA was used to reverse transcribe the first strand cDNA. Fluorescence quantitative PCR was performed using the SYBR Premix ExTaq ∥ kit (Takara) on the CFX96TM Fluorescence Quantitative PCR Detection System (BioRad, Hercules, CA) using gene-specific primers (see Table 1 for quantitative primers).

### (3) Dual Luciferase Reporter Assay

The 3123 bp promoter region of RSB11 was amplified from Zhendao 88 (RSB11-S type variety) and Xiangwanxian 7 (RSB11-R type variety) using primers pSBRR1-LUC-F and pSBRR1-LUC-R (see Table 1 for primers) and then cloned into the multiple cloning site of the pGreenII 0800-LUC vector, respectively, and the Renilla luciferase gene was used as an internal control (Hellens et al., Plant Methods 2005, 1, 13). The two vectors were transfected into rice protoplasts by PEG-mediated transformation (Chern et al., Plant Methods, 2012, 8, 6). Luciferase activity was measured using the dual-luciferase reporter assay system (Promega, E1910). The ratio of LUC to Ren activity was calculated to determine the relative promoter activity (Hellens et al., Plant Methods 2005, 1, 13). Six biological replicates were designed.

### (4) Construction of Recombinant Plant Expression Vector

Construction of complementary vector: The plant binary expression vector pCAMBIA2300 was double-digested with restriction endonucleases BglII and EcoRI, and the linear vector was recovered for later use by electrophoresis. The genomic DNA of the disease-resistant haploid variety Xiangwanxian 7 (XWX7) carrying RSB11-R was used as a template, and the RSB11 genomic promoter region amplification primer pair 2300-ProRSB11-F and 2300-ProRSB11-R (primer sequences are shown in Table 1, and BglII and EcoRI restriction sites and vector recombination linker sequences have been added to the 5′ end, respectively) were used for PCR amplification, and then the obtained RSB11 genomic promoter 3123 bp fragment pRSB11^{XWX7} (SEQ ID NO: 4) was recombined into the cloning vector pCAMBIA2300 using recombinase (Nanjing Novizan), and sequenced to obtain the recombinant vector pCAMBIA2300-pRSB11^{XWX7}. Then, the recombinant vector was double-digested with SmaI and BamHI, and the linear vector was recovered for use by electrophoresis. The genomic DNA of RSB11-S susceptible haplotype variety Dongjin (DJ) was used as a template. Dongjin (DJ) was preserved in our laboratory (Feng et al., Journal of Experimental Botany, 2016, 67, 4241-4253). The RSB11 genome coding region amplification primers 2300-RSB11-F and 2300-RSB11-R (primer sequences are shown in Table 1, and SmaI and BamHI restriction sites and vector recombination linker sequences have been added to the 5′ end, respectively) were used for PCR amplification. Then, the obtained RSB11 genomic DNA coding region 2463 bp fragment CDS-RSB11^{DJ} (SEQ ID NO: 2) was recombined into the cloning vector pCAMBIA2300-pRSB11^{XWX7} using recombinase (Nanjing Novizan), and sequenced to obtain the complementary recombinant vector pRSB11^{XWX7}: cRSB11^{DJ}.

Construction of overexpression vector: The overexpression vector pCAMBIA1390 was digested with PstI, and the linear vector was recovered for later use by electrophoresis. The total plant mRNA of the rice variety Dongjin (DJ) was extracted and used as a template to synthesize the first-strand cDNA. Primers were designed according to the CDS sequence of RSB11 predicted on the NCBI website. The recombination linker sequence on the vector was added to the 5′ end of the front and rear primers. The designed primer pairs RSB11-1390-F and RSB11-1390-R (primer sequences are shown in Table 1, and PstI restriction site and vector recombination linker sequence have been added to the 5′ end) were used to amplify cDNA, electrophoresed, and the fragments were recovered. Then, the obtained 2460 bp containing the full-length CDS sequence of RSB11 gene (SEQ ID NO: 2) was recombined with the pCAMBIA1390 linear vector digested with enzymes using recombinase (Nanjing Novizan), and sequenced to obtain the recombinant vector pUbi:cRSB11^{DJ}.

Construction of gene knockout vector: The RSB11 gene knockout vector was constructed using the CRISPR/Cas9 system. First, the gRNA target sequence was designed and generated, and the target sequence was searched on the genomic sequence of the RSB11 gene. The 18 bp gene-specific spacer sequence of the RSB11 gene (ATACCCTCGCGGTGGGGC (SEQ ID NO: 8)) was cloned into the intermediate vector pOs-sgRNA (Miao et al., Cell Research, 2013, 23: 1233-1236). Then, the sgRNA with the gene-specific spacer sequence was subcloned into the target vector pOs-Cas9 containing the CAS9 expression component using the Gateway LR Clonase II enzyme mixture (Shanghai Yingjun). The specific method is referred to the literature (Miao et al., Cell Research, 2013, 23: 1233-1236) to obtain the RSB11 gene knockout vector pCAS9-RSB11.

TABLE 1
Primer list

| Primer | Primer sequence | |
|---|---|---|
| name | (from 5′ to 3′ ) | SEQ ID NO: |
| qRSB11-F | AGGCTCGATGGTTCACATCAGG | 9 |
| qRSB11-R | AATGCCGCCATCCCAAAGTCTC | 10 |
| pSBRR1- | CCCCCTCGAGGTCGACGTGGGTC | 11 |
| LUC-F | ATGTATGCCGTAA | |
| pSBRR1- | TTGGCGTCTTCCATGGAGAAGT | 12 |
| LUC-R | AGGTGTTGTTGGGAC | |
| 2300- | CCATGGTCGATCGACAGATCTA | 13 |
| ProRSB11- | TGCGTCATGCTGTGGGTC | |
| F | | |
| 2300- | CCGGGTACCGAGCTCGAATTCT | 14 |
| ProRSB11- | GTGAGAAGTAGGTGTTGTTGGG | |
| R | AC | |
| 2300- | TCGAGCTCGGTACCCGGGATGT | 15 |
| RSB11-F | CTCCACTCCACACCCA | |
| 2300- | GGTCGACTCTAGAGGATCCTTA | 16 |
| RSB11-R | GAACCCGACTAAAATCC | |
| RSB11- | TCTGCACTAGGTACCTGCAGAT | 17 |
| 1390-F | GTCTCCACTCCACACCCA | |
| RSB11- | ATGGATCCGTCGACCTGCAGGA | 18 |
| 1390-R | ACCCGACTAAAATCC | |
| P1 | ACGCGTAGTGCTGTCACTTG | 19 |
| P2 | TGATGATGGCAAGGCTACTG | 20 |
| P3 | CTAGCTAGAGTCGAGAATTCAGT | 21 |

Note:
The underlined sequence indicates the sequence on the vector.

### II. Results and Analysis

### 1. Identification of RSB11 Gene

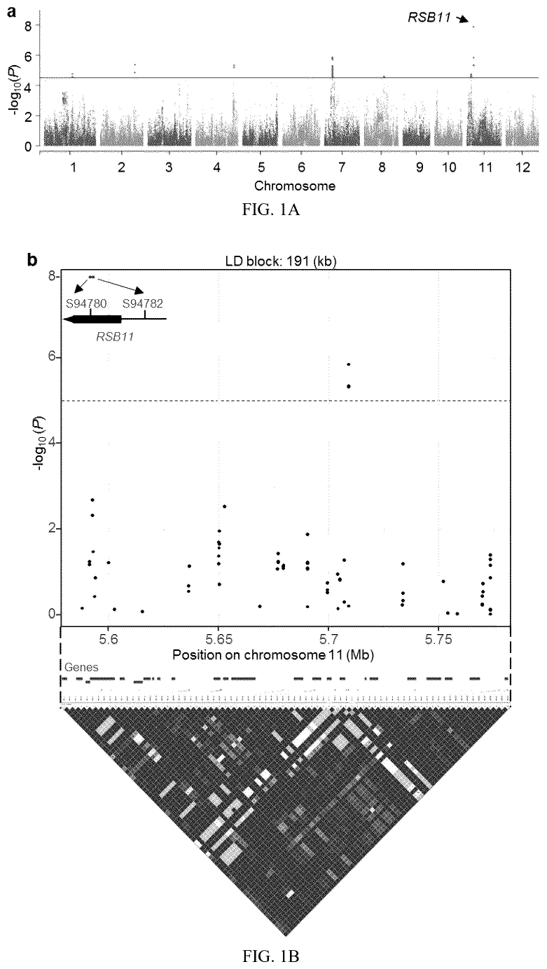

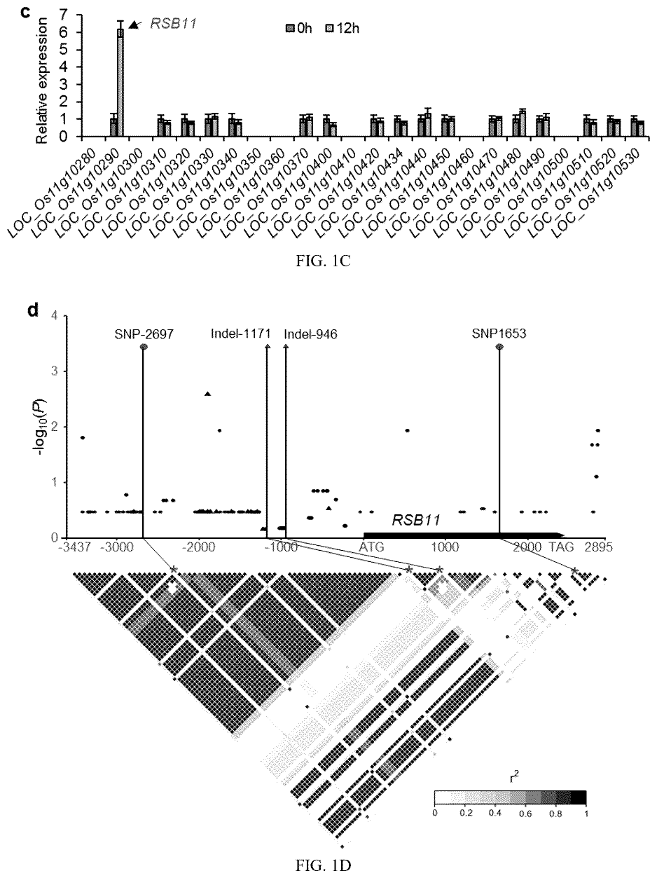

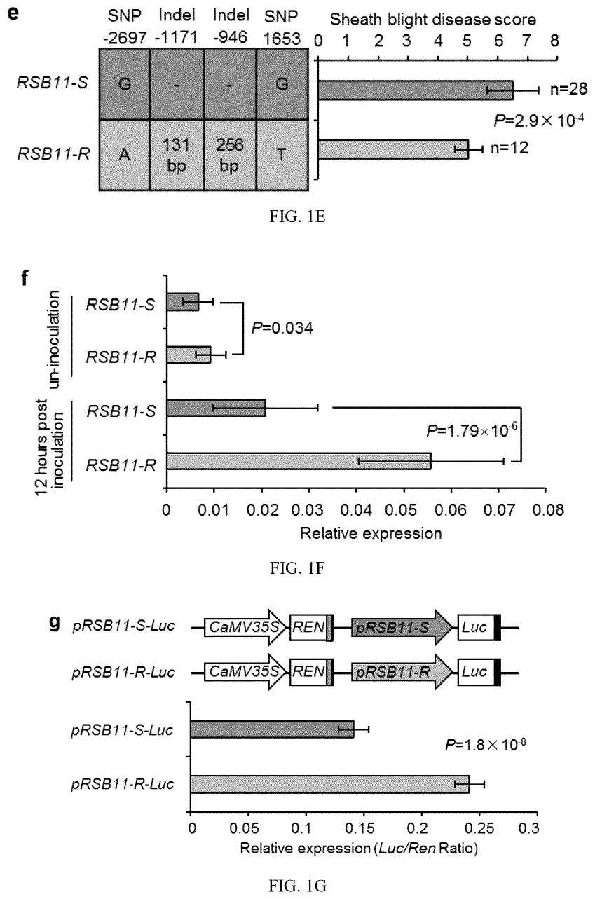

In order to study the resistance of rice to sheath blight, the present invention resequenced and identified the sheath blight resistance of 178 promoted rice varieties from different regions of China, Japan and South Korea, and identified 48 SNIP sites significantly associated with sheath blight resistance using GWAS (FIG. 1A). The two SNIPs with the strongest association were SNIP94780 and SNIP94782 on chromosome 11, which were 3.9 kb apart and contributed 16.82% to sheath blight resistance. These two sites were located in an LD block with a physical distance of 191 kb, which contained 24 genes (FIG. 1B). We found that among these genes, only gene LOC_Os11g10290 was strongly induced to express by sheath blight infection (FIG. 1C). Combined with the fact that the two SNPs with the highest association significance were also located in the promoter and coding region of LOC_Os11g10290, respectively (FIG. 1B), we inferred that this gene was likely related to sheath blight resistance and named it RSB11 (Resistance gene to sheath blight on chromosome11). The RSB11 gene encodes a lectin receptor kinase protein (LecRLK). The RSB11 gene in the wild-type Nipponbare has the nucleotide sequence shown in SEQ ID NO: 2, and the protein sequence it encodes is SEQ ID NO: 1.

We then sequenced the RSB11 gene in 20 susceptible varieties with disease levels greater than 6.5 and 20 relatively resistant varieties with disease levels less than 5.5 (Table 2). The sequencing interval included 3341 bp of the promoter region, 96 bp of the 5′ non-coding region, 2463 bp of the coding region, 143 bp of the 3′ non-coding region, and 150 bp downstream of the 3′ non-coding region. The sequencing results and the disease levels of the varieties were used to further conduct association analysis based on the RSB11 gene. The results showed that an SNP site (SNP94780) in the coding region and three SNP/Indel sites (SNP94782, Indel1171, and Indel946) in the promoter region were most significantly associated with sheath blight resistance (FIG. 1D). Among them, SNP94782 is an SNP in the rice genome, corresponding to the 515th nucleotide of SEQ ID NO: 4, which is T or G; Indel1171 is a deletion variation in the rice genome, corresponding to The nucleotides 2005-2135 (131 bp) of SEQ ID NO: 4 are deleted or not deleted; Indel946 is a deletion variation in the rice genome, corresponding to the nucleotides 2231-2486 (256 bp) of SEQ ID NO: 4, which are deleted or not deleted; SNP94780 is an SNP in the rice genome, corresponding to the nucleotide 1653 of SEQ ID NO: 2, which is A or G. Based on these four variant sites, RSB11 alleles were divided into two haplotypes: RSB11-S, susceptible to sheath blight, and RSB11-R, resistant to sheath blight (FIG. 1E). The homozygous rice genotype corresponding to haplotype RSB11-R was more resistant to sheath blight than the homozygous rice genotype corresponding to haplotype RSB11-S. Haplotype RSB11-R was: SNP94782 was T, Indel1171 was non-deletion, Indel946 was non-deletion, and SNP94780 was A. Haplotype RSB11-S was: SNP94782 was G, Indel1171 was deletion, Indel946 was deletion, and SNP94780 was G. Since SNP94780 located in the coding region does not cause amino acid changes (the RSB11 gene sequence with SNP94782 as G is shown in SEQ ID NO: 3, coding the protein shown in SEQ TD NO: 1) and other three mutation sites are located in the promoter region, so these three mutations may affect the expression level of RSB11. We used the fluorescence quantitative primers qRSB11-F and qRSB11-R of RSB11 (Table 1) to randomly detect the expression levels of RSB11 in 20 RSB11-S types rice (Xiangwanxian 1, Hongwan 5202, Shenglixian 44, Nanhua 11, Ewan 5, Yangdao 4, Zhendao 88, Lianjing 6, Wulingjing 1, Early-maturing Fragrant Black Rice, Xiangnong 15, Aizhe Jiuxuan, Zhaiyeqing 8, Mabaojin, Hexi 41, Wenguangqing, Zhaierdao, Youliudao, Tie 9868, Nanyuandao) and 12 RSB11-R types rice (Yangdao 3, Jiahe 218, Guichao 2, Nante, Huangsi Guizhan, Xiang'ai Zao 3, Xiangzhou 7, Huazhong 1, Te San Ai 2, Yuegui 146, Xiangwanxian 7, Siqingwanxian) before and after inoculation with sheath blight pathogen RH-9. We found that the expression level of RSB11-R was higher than that of RSB11-S before inoculation (P=0.034), and 12 hours after inoculation, RSB11-R was more strongly induced than RSB11-S (P=1.79×10−6), reaching a level 2.7 times that of RSB11-S (FIG. 1F).

TABLE 2
Varieties used for RSB11 gene sequencing

| Field No. | Variety Name | disease levels | Field No. | Variety Name | disease levels |
|---|---|---|---|---|---|
| P248 | Ewan 5 | 5.48 ± 0.53 | P232 | Xiangwan Xianxian 1 | 6.85 ± 0.23 |
| P249 | Yangdao 3 | 4.15 ± 0.33 | P234 | Hongwan 5202 | 7.07 ± 0.41 |
| P266 | Jiahe 218 | 5.22 ± 1.38 | P242 | Shengli Xianxian 44 | 7.05 ± 0.84 |
| P275 | Guichao 2 | 5.43 ± 0.54 | P245 | Nanhua 11 | 6.92 ± 0.19 |
| P279 | Nante | 4.82 ± 0.59 | P250 | Yangdao 4 | 6.82 ± 0.25 |
| P282 | Huangsi Guizhan | 5.5 ± 0.45 | P260 | Zhendao 88 | 6.83 ± 0.24 |
| P289 | Xiangnong 15 | 5.48 ± 0.44 | P264 | Lianjing 6 | 6.82 ± 0.25 |
| P294 | Xiangai Zao 3 | 5.47 ± 0.61 | P267 | Wulingjing 1 | 6.83 ± 0.24 |
| P299 | Xiangzhou 7 | 4.57 ± 0.45 | P283 | Early-maturing fragrant | 6.9 ± 0.56 |
| | | | | black rice | |
| P308 | Huazhong 1 | 5.2 ± 0.55 | P298 | Dwarf Zhejiang 9 | 7.27 ± 0.52 |
| | | | | selection | |
| P338 | Tesanai 2 | 5.42 ± 0.66 | P314 | Zhaiyeqing 8 | 7.1 ± 0.61 |
| P339 | Yuegui 146 | 4.67 ± 0.61 | P326 | Mabaojin | 6.83 ± 0.62 |
| P349 | Xiangwan Xianxian 7 | 4.47 ± 0.43 | P327 | Qisi Zao 3 | 7.17 ± 0.65 |
| P360 | Liaojing 371 | 4.64 ± 0.49 | P368 | Hexi 41 | 7.47 ± 0.51 |
| P364 | Zhongzuo 332 | 5.2 ± 0.86 | P388 | Zhongan Dao | 6.87 ± 0.22 |
| P563 | Shufeng 101 | 5.14 ± 1.29 | P562 | Nanjing 11 | 7.2 ± 0.65 |
| P565 | Qingai Zao 5 | 5.23 ± 0.75 | P568 | Wenguangqing | 7.27 ± 0.59 |
| P566 | Siqing Wanxian | 5.37 ± 0.67 | P571 | Zhaier Dao | 7.1 ± 1.26 |
| P582 | Tie 9868 | 5.2 ± 0.37 | P572 | Youliu Dao | 7.07 ± 0.78 |
| P591 | Nanyuandao | 5.3 ± 0.46 | P575 | Yuexinzhan 5 | 6.87 ± 0.23 |

We further constructed luciferase reporter gene vectors driven by the RSB11 promoter of RSB11-S type variety Zhendao 88 and RSB11-R type variety Xiangwanxian 7 (XWX7), respectively, and conducted transient expression experiments in rice protoplasts, confirming that the RSB11-R promoter is more effective than the RSB11-S promoter in promoting the expression of the reporter gene (FIG. 1G). Wherein, the nucleotide sequence of the RSB11-R promoter is shown in SEQ ID NO: 4.

The above results indicate that the promoter variation that determines the expression level of RSB11 is the reason for the difference in the resistance level of sheath blight between RSB11-R and RSB11-S rice varieties.

### 2. Obtaining Transgenic Plants and Phenotypic Identification

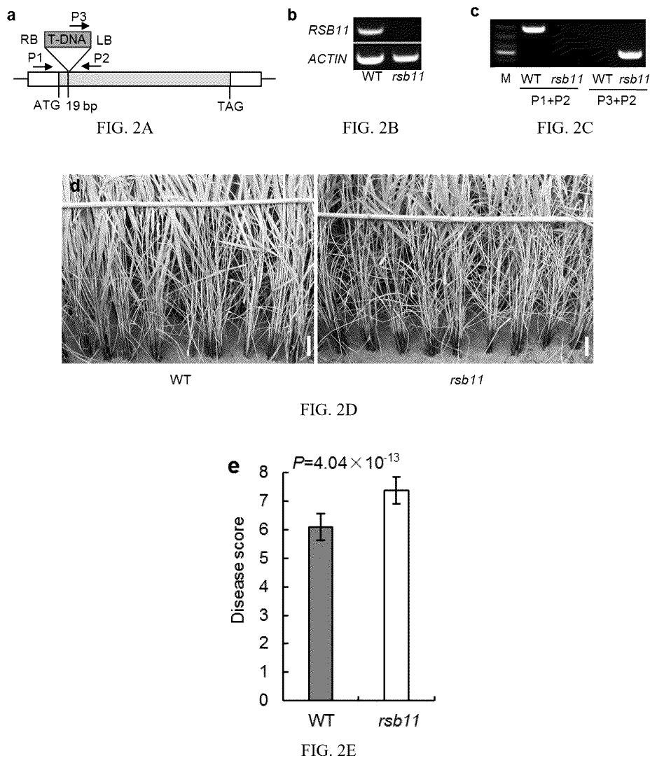

To verify whether RSB11 is involved in regulating rice sheath blight resistance, we first identified a RSB11 T-DNA insertion mutant rsb11 in the RSB11-S rice variety Dongjin (DJ) background. rsb11 was identified from a T-DNA insertion mutant library and numbered 3D-50196L (Jeon et al., 2000; http://orygenesdb.cirad.fr/). Molecular markers P1, P2, and P3 (Table 1) confirmed that the T-DNA was inserted at the 19 bp downstream of ATG, resulting in the non-expression of the RSB11 gene in the mutant (FIGS. 2A-2C). In the field inoculation experiment, we found that compared with the wild type WT (i.e. Dongjin (DJ), disease level 6.09), the rsb11 mutant (disease level 7.38) was more susceptible to sheath blight (FIGS. 2D-2E). Then, the constructed complementation vector pRSB11^{XWX7}:cRSB11^{DJ} (RSB11 promoter in XWX7 drives RSB11 gene coding region in DJ) was transformed into mutant rsb11 by agrobacterium-mediated rice genetic transformation method (Hiei et al., The plant journal, 6, 217-282). The overexpression vector pUbi:cRSB11^{DJ} (maize ubiquitin Ubi promoter drives RSB11 gene coding region in Dongjin (DJ)) was transformed into Dongjin (DJ), and 15 complementation lines and 20 overexpression lines of TO generation were obtained. We finally determined two stable complementation lines (Com-1 and Com-2) and three overexpression lines (RSB11-OE1, RSB11-OE2 and RSB11-OE3) for the next experiment.

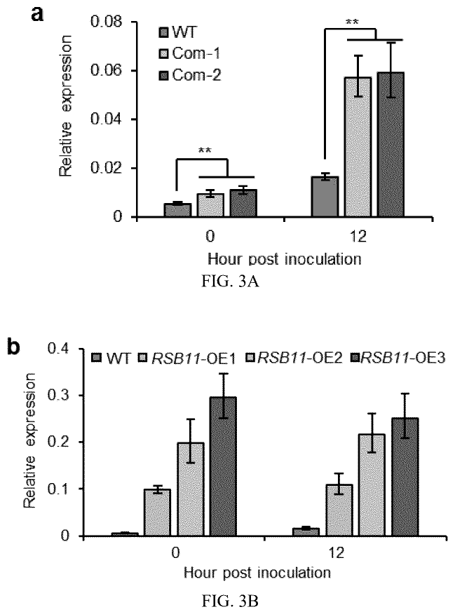

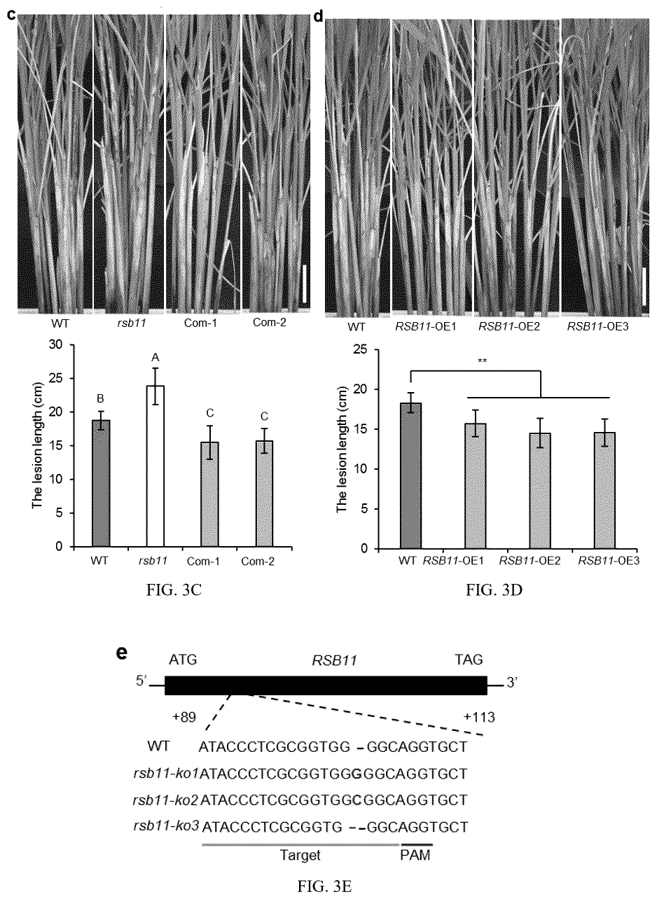

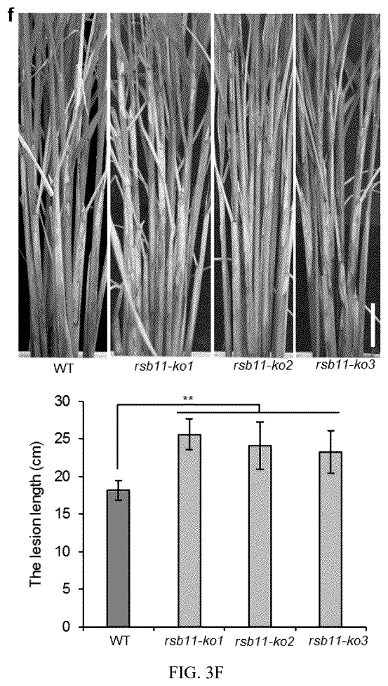

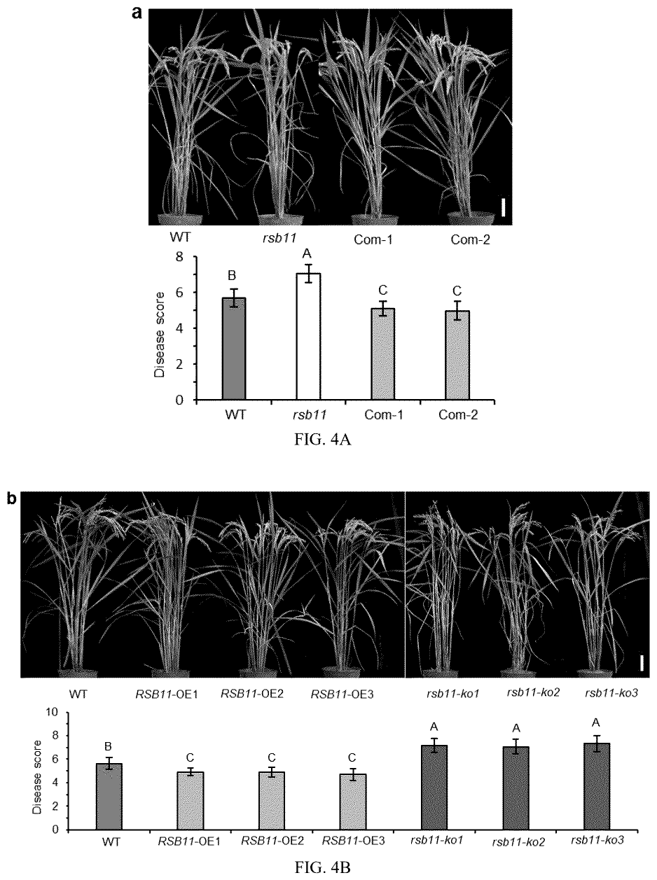

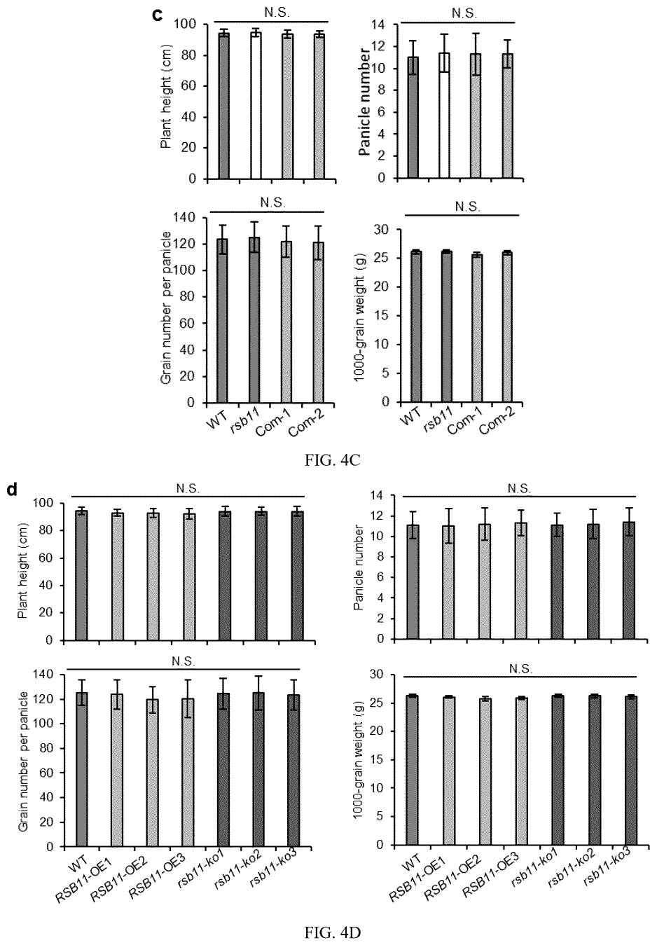

We first detected the RNA expression levels of complementation lines and overexpression lines by qRT-PCR. Compared with WT plants, both complementation lines showed significantly enhanced expression levels, reaching about 3 times the level of Dongjin (DJ) after inoculation with sheath blight pathogen RH-9, indicating that the RSB11-R promoter in XWX7 can indeed promote the expression of RSB11 more strongly than the RSB11-S promoter in Dongjin (DJ) (FIG. 3A). The three overexpression lines also clearly showed much higher transcription levels than WT (FIG. 3B). Next, we identified their resistance to sheath blight in the greenhouse, and the lesion lengths of the two complementation lines (15.49 and 15.71 cm) were significantly shorter than those of WT (18.75 cm) and the rsb11 mutant (23.84 cm) (FIG. 3C). The lesion lengths of the three overexpression lines were close to those of the complementation lines and significantly shorter than those of WT (FIG. 3D). In addition, we also knocked out RSB11 in Dongjin (DJ) using the CRISPR/Cas9 gene editing system and obtained three knockout lines (rsb11-ko1, rsb11-ko2, and rsb11-ko3) carrying different sequence variations (FIG. 3E). The knockout lines were all more susceptible to the disease than the WT (FIG. 3F). We also identified the sheath blight resistance of these transgenic lines in the field and obtained results consistent with those in the greenhouse (FIGS. 4A-4B). It is worth noting that compared with the WT, the main agronomic traits of the complementation line, overexpression line, and knockout line (for detection methods, see step 1 (2) in Example 2) showed almost no significant changes (FIGS. 4C-4D). In summary, these results confirm that RSB11 positively regulates rice resistance to sheath blight.

### Example 2. Disease-Resistant Haplotype RSB11-R Improves Resistance of Japonica Rice Varieties to Sheath Blight

### I. Materials and Methods

### (1) Yield Loss Test Due to Sheath Blight

The experiment was conducted with reference to the method reported by previous researchers (Zuo Shimin et al., Chinese Rice Science, 2007, 21, 136-142). The near-isogenic line NIL-RSB11-R and its control Taijing 394 were planted in the experimental field with the same fertility level. Two conditions of mild disease and severe disease were set. Field stalks were built between the plots under different experimental conditions. The experiment was repeated 3 times. Each experimental plot was planted with 10 rows and 40 holes per row. Under the mild disease condition, the pesticide thiophanate-methyl for preventing and controlling sheath blight was sprayed in the late tillering stage to prevent sheath blight from occurring. Under the severe disease condition, referred to the sheath blight inoculation method in Example 1, 5 stems were inoculated per plant to ensure full disease. When the plants were fully mature, the sheath blight disease level of each experimental plot was investigated. Plants in the middle area of 1.32 m² in each experimental plot were selected to measure yield and other agronomic traits.

### (2) Agronomic Trait Survey

The materials used for the agronomic trait survey were all planted in the rice experimental field on the campus of Yangzhou University. According to the agronomic trait survey method developed by the International Rice Research Institute (Standard Evaluation System for Rice, 4th ed. 2022, International Rice Research Institute, Los Banos, Philippines, Pages 15-16), the traits include plant height, growth period, number of effective panicles, number of grains per panicle, thousand-grain weight, fruiting rate, plot yield, amylose content and chalky grain rate, etc.

### II. Results and Analysis

### 1. The Disease-Resistant Haplotype RSB11-R can Significantly Improve the Resistance of Japonica Rice Varieties to Sheath Blight

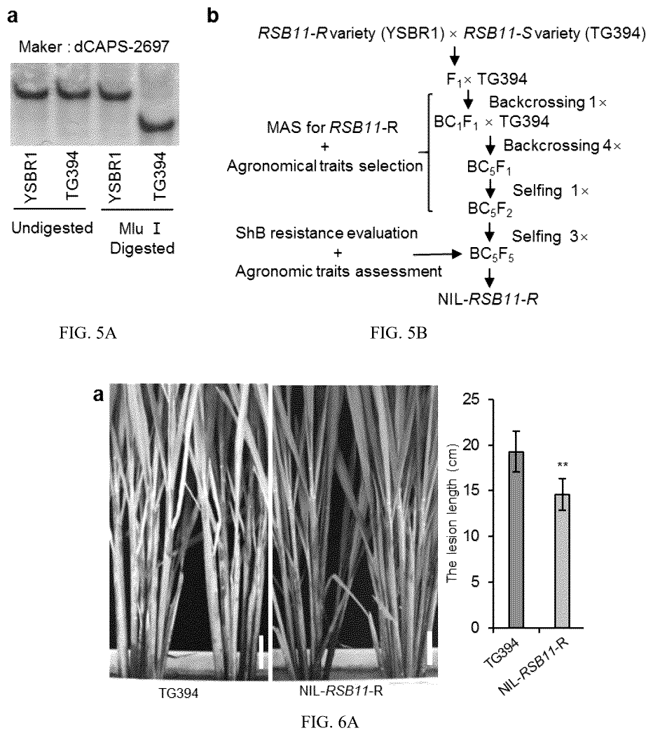

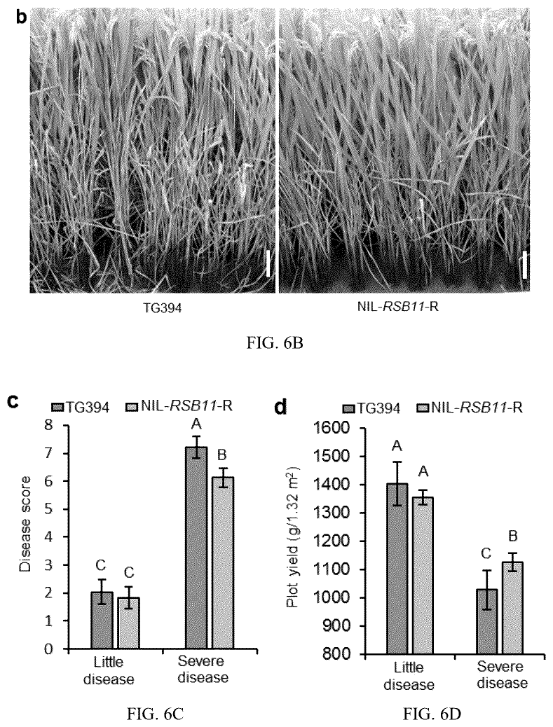

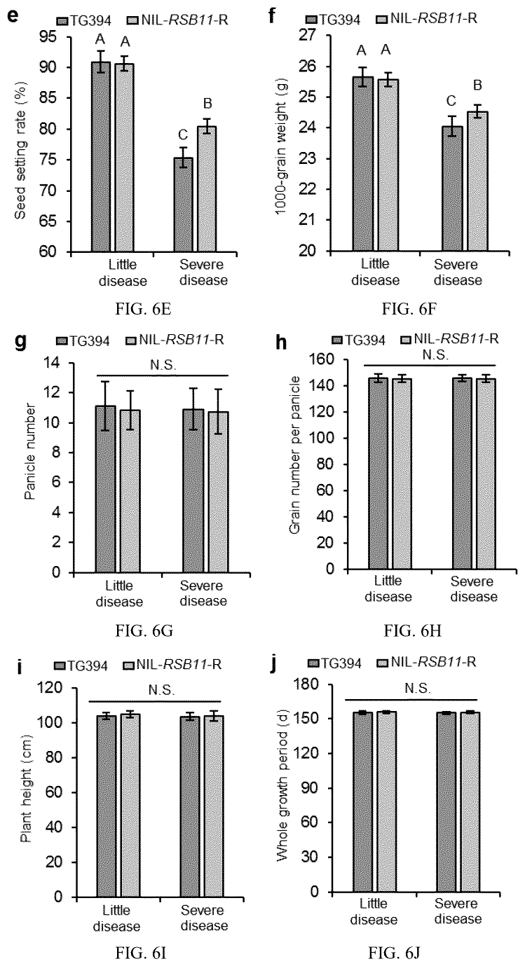

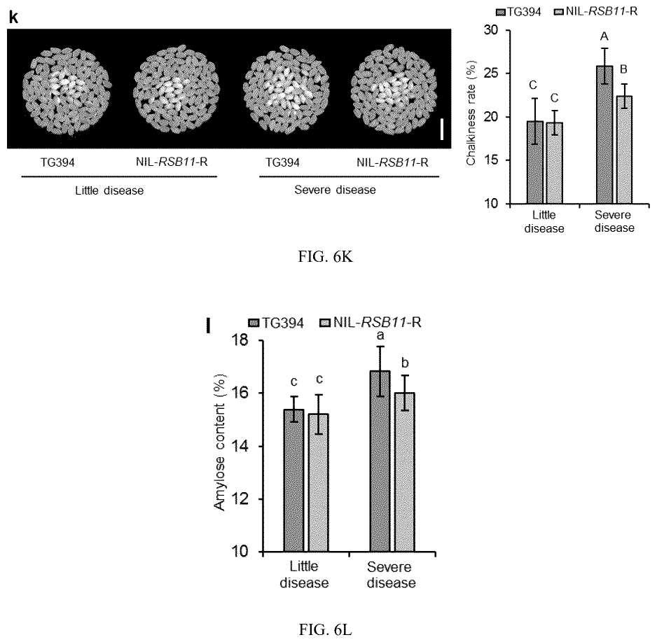

For the key difference SNP site SNP94782 in the gene promoter region between the disease-resistant haplotype RSB11-R and the susceptible haplotype RSB11-S in Example 1, we designed a functional dCAPS molecular marker dCAPS782 that specifically distinguishes the RSB11 gene resistant and susceptible haplotypes. Its front primer dCAPS782-F has the nucleotide sequence shown in SEQ ID NO: 5, and the rear primer dCAPS782-R has the nucleotide sequence shown in SEQ ID NO: 6. This marker can amplify a 154 bp band in both RSB11 resistant and susceptible haplotype varieties (SEQ ID NO: 7, wherein K represents T or G), after the amplified band was digested with restriction endonuclease MluI, the fragment of the disease-resistant haploid variety could not be cut and the size remained unchanged, while the susceptible haploid variety could be cut into two segments of 130 bp and 24 bp in size (FIG. 5A). The marker-assisted selection breeding process is shown in FIG. 5B. The rice variety YSBR1 carrying the disease-resistant haplotype RSB11-R is used as the donor parent, and the Jiangsu popular japonica rice variety Taijing 394 (TG394) carrying the susceptible haplotype RSB11-S is used as the recurrent parent. Continuous backcrossing breeding is carried out, and the above-mentioned functional molecular marker dCAPS782 is used for marker-assisted selection. Finally, the near-isogenic line NIL-RSB11-R containing RSB11-R is obtained in the BC5F5 generation (FIG. 5B). The results of the inoculation identification of sheath blight resistance showed that the sheath blight resistance of the near-isogenic line NIL-RSB11-R was significantly enhanced compared with TG394 (FIG. 6A).

To evaluate the breeding potential of RSB11-R, we conducted field yield loss rate tests on NIL-RSB11-R and TG394 under two sheath blight disease conditions: one was mild disease; the other was severe disease (FIGS. 6B-6L). We found that under mild disease conditions, the disease level of TG394 was 2.03, and NIL-RSB11-R and TG394 did not show significant differences in disease level (FIG. 6C). In addition, there were no significant differences in their main agronomic traits, yield-related traits, and quality-related traits, indicating that the introduction of RSB11-R had no adverse effects on rice development, yield, and quality (FIGS. 6D-6L). Under severe disease conditions, the disease level of NIL-RSB11-R was 6.13, which was significantly lower than that of TG394 (7.22), indicating that RSB11-R had a good disease resistance effect in the field trial (FIGS. 6B-6C). Under severe disease conditions, although the plant yield and quality of TG394 and NIL-RSB11-R decreased significantly, which mainly reflected in the decrease of fruit setting rate and 1000-grain weight, and the increase of chalky grain rate and amylose content (FIGS. 6D-6I), the yield loss of NIL-RSB11-R was significantly lower than that of TG394. The yield of TG394 decreased from 1404.0 g/1.32 m2 to 1028.5 g/1.32 m2, a decrease of 26.75%, and the yield of NIL-RSB11-R decreased from 1354.3 g/1.32 m2 to 1126.6 g/1.32 m2, a decrease of 16.82%. Therefore, RSB11-R reduced the severity of sheath blight and recovered 9.54% of the yield loss (FIG. 6D). Further analysis found that the recovered yield loss was mainly reflected in two yield components: fruiting rate (6.75%) and 1000-grain weight (2.00%) (FIGS. 6E-6F). NIL-RSB11-R also had significantly lower quality loss than TG394, including lower chalky grain rate and amylose content, indicating that RSB11-R was also beneficial to quality improvement under severe disease conditions (FIGS. 6K-6L). The above results show that the superior natural allele RSB11-R has great application potential in the breeding of japonica rice for resistance to sheath blight.

### INDUSTRIAL APPLICATION

The method of cultivating rice varieties with improved resistance to sheath blight according to the present invention can be to introduce the superior natural allele RSB11-R of RSB11 into conventional japonica rice varieties by hybridization, backcrossing and combining with marker-assisted selection (MVAS) technology to obtain a new conventional rice line with enhanced resistance to sheath blight. The method can also reduce rice yield loss at the same time.

The invention disclosed a quantitative gene RSB11 for resistance to sheath blight cloned by genome-wide association analysis, which encodes a lectin receptor kinase protein. Three variations in the promoter region of RSB11 increase its expression level and resistance to sheath blight, thereby producing an excellent natural allele RSB11-R. Compared with the wild type, the expression of RSB11 gene increases, and the resistance to sheath blight is enhanced; while after the RSB11 gene is knocked out, the resistance to sheath blight is weakened. Transforming RSB11-R into susceptible haploid japonica rice varieties through molecular marker-assisted selection can improve its resistance to sheath blight, without affecting basic agronomic traits, and can recover 9.54% of yield loss under severe disease conditions, indicating that RSB11-R has important application value in molecular breeding of rice disease resistance. The RSB11 gene and the encoded protein of the invention are of great significance for cultivating rice varieties resistant to sheath blight and reducing rice yield losses.

What is claimed is:

1. A DNA molecule having the nucleotide sequence shown in SEQ ID NO: 4.

2. A recombinant vector, expression cassette, transgenic cell line or recombinant bacteria containing the DNA molecule of claim 1.

3. A use of the DNA molecule of claim 1 as a promoter in enhancing an expression of a target gene in a plant.

4. The use of claim 3, wherein the target gene is a nucleic acid molecule capable of expressing an RSB11 protein.

5. The use of claim 4, wherein the RSB11 protein is any one of the following:
(A1) a protein having the amino acid sequence of SEQ ID NO: 1;
(A2) a protein derived from rice with a same function of SEQ ID NO: 1 and having an amino acid sequence obtained by replacing and/or deleting and/or adding one or more amino acid residues of the amino acid sequence shown in SEQ ID NO: 1;
(A3) a protein having 99% or more, 95% or more, 90% or more, 85% or more, or 80% or more identity with the amino acid sequence defined in any one of (A1)-(A2) and derived from rice with a same function of the amino acid sequence defined in any one of (A1)-(A2);
(A4) a fusion protein obtained by connecting a protein tag to an N-terminus and/or C-terminus of the protein of any one of (A1)-(A3).

6. A use of the DNA molecule of claim 1 in any one of the following (a1)-(a2):
(a1) improving plant resistance to sheath blight;
(a2) improving plant resistance to Rhizoctonia solani Kühn.

7. The use of claim 6, wherein in the use, the DNA molecule initiates an expression of a target gene in a plant, and the target gene is a nucleic acid molecule capable of expressing an RSB11 protein.

8. The use of claim 7, wherein the RSB11 protein is any one of the following:
(A1) a protein having the amino acid sequence of SEQ ID NO: 1;
(A2) a protein derived from rice with a same function of SEQ ID NO: 1 and having an amino acid sequence obtained by replacing and/or deleting and/or adding one or more amino acid residues of the amino acid sequence shown in SEQ ID NO: 1;
(A3) a protein having 99% or more, 95% or more, 90% or more, 85% or more, or 80% or more identity with the amino acid sequence defined in any one of (A1)-(A2) and derived from rice with a same function of the amino acid sequence defined in any one of (A1)-(A2);
(A4) a fusion protein obtained by connecting a protein tag to an N-terminus and/or C-terminus of the protein of any one of (A1)-(A3).

9. A primer pair consisting of two single-stranded DNA molecules shown in SEQ ID NO: 5 and SEQ ID NO: 6.

10. A kit containing the primer pair of claim 9, wherein the kit further contains a restriction endonuclease MluI.

11. A use of the DNA molecule of claim 1, a primer pair, or a kit in plant breeding, wherein the primer pair consisting of two single-stranded DNA molecules shown in SEQ ID NO: 5 and SEQ ID NO: 6, the kit contains the primer pair and a restriction endonuclease MluI.

12. Any of the following uses:
M5: a use of a substance for detecting polymorphism or genotype of four variant sites SNP94782, Indel1171, Indel946 and SNP94780 in identifying or assisting in identifying rice resistance to sheath blight; wherein SNP94782 is an SNP in a rice genome, corresponding to a 516th nucleotide of SEQ ID NO: 4, wherein SNP94782 is T or G; Indel1171 is a deletion variation in the rice genome, corresponding to 2005-2135th nucleotides of SEQ ID NO: 4, wherein Indel1171 is deletion or non-deletion; Indel946 is a deletion variation in the rice genome, corresponding to nucleotides 2231-2486 of SEQ ID NO: 4, wherein Indel946 is deletion or non-deletion; SNP94780 is an SNP in the rice genome, corresponding to nucleotide 1653 of SEQ ID NO: 2 or SEQ ID NO: 3, wherein SNP94780 is A or G;
M6: a use of a substance for detecting a haplotype in identifying or assisting in identifying rice resistance to sheath blight; the haplotype is a polymorphism or genotype combination of the four variable sites SNP94782, Indel1171, Indel946 and SNP94780 in M5 on the rice genome;
M7: a use of a substance for detecting the polymorphism or genotype of SNP94782 in M5 in identifying or assisting in identifying rice resistance to sheath blight;
M8: a use of the primer pair of claim 9 or a kit in detecting the polymorphism or genotype of SNP94782 in M5, wherein the kit contains the primer pair and a restriction endonuclease MluI;
M9: a use of the primer pair of claim 9 or the kit in identifying or assisting in identifying rice resistance to sheath blight.

13. The use of claim 3, wherein the plant is a monocot or a dicot.

14. The use of claim 13, wherein the monocot is a grass plant.

15. The use of claim 14, wherein the grass plant is a rice plant.

16. The use of claim 15, wherein the rice plant is rice.

17. Any of the following methods:
Q5: a method for identifying or assisting in identifying rice resistance to sheath blight, comprising the following steps (C1) or (C2):
(C1) detecting the haplotype in M6 of claim 12 in a genome of a rice to be tested, and determining a resistance of the rice to be tested to sheath blight according to a haplotype of the rice to be tested as follows: a resistance of a homozygous genotype rice corresponding to a haplotype RSB11-R to sheath blight is stronger or is a candidate for being stronger than a homozygous genotype rice corresponding to a haplotype RSB11-S; the haplotype RSB11-R is: SNP94782 is T, Indel1171 is non-deletion, Indel946 is non-deletion, and SNP94780 is A; the haplotype RSB11-S is: SNP94782 is G, Indel1171 is deletion, Indel946 is deletion, and SNP94780 is G;
(C2) detecting SNP94782 in M5 of claim 12 in the genome of the rice to be tested, and determining the resistance of the rice to be tested to sheath blight according to the genotype of SNP94782 of the rice to be tested as follows: a resistance of a rice with the genotype of SNP94782 being TT to sheath blight is stronger than or is a candidate to be stronger than a resistance of a rice with the genotype of SNP94782 being GG to sheath blight;
Q6: a method for cultivating a rice variety with improved resistance to sheath blight, comprising the following steps: selecting a rice variety identified by the method of Q5 with a relatively strong resistance to sheath blight as a donor parent, and selecting a rice variety identified by the method of Q5 with a relatively weak resistance to sheath blight but with expected agronomic traits as a recurrent parent, and obtaining the rice variety with improved resistance to sheath blight and the expected agronomic traits through continuous backcrossing.

18. The method of claim 17, wherein in step (C2) of the method of Q5, using the primer pair or the kit to detect the genotype of SNP94782 in the genome of the rice to be tested.

19. The method of claim 18, wherein taking a genome DNA of the rice to be tested as a template, and amplifying with the primer pair, if a target fragment of 154 bp is obtained, as shown in SEQ ID NO: 7, and a 28th position is homozygous T, the genotype of SNP94782 in the genome of the rice to be tested is TT; if the target fragment of 154 bp is obtained, as shown in SEQ ID NO: 7, and the 28th position is homozygous G, the genotype of SNP94782 in the genome of the rice to be tested is GG.

20. The method of claim 18, wherein taking a genome DNA of the rice to be tested as a template, amplifying with the primer pair, and digesting an amplified product completely with MluI, if a digestion product is 154 bp, the genotype of SNP94782 in the genome of the rice to be tested is TT; if the digestion products are 130 bp and 24 bp, the genotype of SNP94782 in the genome of the rice to be tested is GG.

21. A use of an RSB11 protein or a related biological material thereof in any one of the following:
P1, regulating plant resistance to sheath blight;
P2, regulating plant resistance to Rhizoctonia solani Kühn;
the RSB11 protein is any one of the following:
(A1) a protein having the amino acid sequence of SEQ ID NO: 1;
(A2) a protein derived from rice with a same function of SEQ ID NO: 1 and having an amino acid sequence obtained by replacing and/or deleting and/or adding one or more amino acid residues of the amino acid sequence shown in SEQ ID NO: 1;
(A3) a protein having 99% or more, 95% or more, 90% or more, 85% or mor, or 80% or more identity with the amino acid sequence defined in any one of (A1)-(A2) and derived from rice with a same function of the amino acid sequence defined in any one of (A1)-(A2);
(A4) a fusion protein obtained by connecting a protein tag to an N-terminus and/or C-terminus of the protein of any one of (A1)-(A3),
the related biological material is a nucleic acid molecule capable of expressing the RSB11 protein, or an expression cassette, a recombinant vector, a recombinant microorganism or a transgenic cell line containing the nucleic acid molecule.

22. The use of claim 21, wherein in a plant, an expression level and/or activity of the RSB11 protein increases, and the resistance to sheath blight and/or resistance to Rhizoctonia solani Kühn increases;
in the plant, the expression level and/or activity of the RSB11 protein decreases, and the resistance to sheath blight increases and/or resistance to Rhizoctonia solani Kühn decreases.

23. The use of claim 21, wherein the nucleic acid molecule capable of expressing the RSB11 protein is any one of the following:
(B1) a DNA molecule shown in SEQ ID NO: 2 or SEQ ID NO: 3;
(B2) a DNA molecule hybridizing with the DNA molecule defined in (B1) under stringent conditions and encoding the RSB11 protein;
(B3) a DNA molecule having 99% or more, 95% or more, 90% or more, 85% or more, or 80% or more identity with any one of the DNA sequences defined in (B1)-(B2) and encoding the RSB11 protein.

24. Any of the following uses:
M3: a use of a substance capable of increasing an expression level and/or activity of an RSB11 protein in a plant in any one of the following (a1)-(a2);
(a1) improving plant resistance to sheath blight;
(a2) improving plant resistance to Rhizoctonia solani Kühn;
M4: a use of a substance capable of reducing the expression level and/or activity of the RSB11 protein in the plant in any one of the following (b1)-(b2):
(b1) reducing plant resistance to sheath blight;
(b2) reducing plant resistance to Rhizoctonia solani Kühn.
the RSB11 protein is any one of the following:
(A1) a protein having the amino acid sequence of SEQ ID NO:1;
(A2) a protein derived from rice with a same function of SEQ ID NO: 1 and having an amino acid sequence obtained by replacing and/or deleting and/or adding one or more amino acid residues of the amino acid sequence shown in SEQ ID NO: 1;
(A3) a protein having 99% or more, 95% or more, 90% or more, 85% or more, or 80% or more identity with the amino acid sequence defined in any one of (A1)-(A2) and derived from rice with a same function of the amino acid sequence defined in any one of (A1)-(A2);
(A4) a fusion protein obtained by connecting a protein tag to an N-terminus and/or C-terminus of the protein of any one of (A1)-(A3).

25. The use of claim 21, wherein the plant is a monocot or a dicot.

26. The use of claim 25, wherein the monocot is a grass plant.

27. The use of claim 26, wherein the grass plant is a rice plant.

28. The use of claim 27, wherein the rice plant is rice.

29. Any of the following methods:
Q1: a method for cultivating a plant with enhanced resistance to sheath blight and/or enhanced resistance to Rhizoctonia solani Kühn, comprising a step of increasing an expression level and/or activity of an RSB11 protein in a recipient plant;
Q2: a method for cultivating a plant with reduced resistance to sheath blight and/or reduced resistance to Rhizoctonia solani Kühn, comprising a step of reducing the expression level and/or activity of the RSB11 protein in a recipient plant;
Q3: a method for cultivating a transgenic plant with enhanced resistance to sheath blight and/or enhanced resistance to Rhizoctonia solani Kühn, comprising the following steps: introducing a nucleic acid molecule capable of expressing the RSB11 protein into a recipient plant to obtain the transgenic plant; the transgenic plant has enhanced resistance to sheath blight and/or enhanced resistance to Rhizoctonia solani Kühn compared with the recipient plant;
Q4: a method for cultivating a transgenic plant with reduced resistance to sheath blight and/or reduced resistance to Rhizoctonia solani Kühn, comprising the following steps: inhibiting the expression of the nucleic acid molecule capable of expressing the RSB11 protein in a recipient plant to obtain the transgenic plant; the transgenic plant has reduced resistance to sheath blight and/or reduced resistance to Rhizoctonia solani Kühn compared with the recipient plant;
the RSB11 protein is any one of the following:
(A1) a protein having the amino acid sequence of SEQ ID NO: 1;
(A2) a protein derived from rice with a same function of SEQ ID NO: 1 and having an amino acid sequence obtained by replacing and/or deleting and/or adding one or more amino acid residues of the amino acid sequence shown in SEQ ID NO: 1;
(A3) a protein having 99% or more, 95% or more, 90% or more, 85% or more, or 80% or more identity with the amino acid sequence defined in any one of (A1)-(A2) and derived from rice with a same function of the amino acid sequence defined in any one of (A1)-(A2);
(A4) a fusion protein obtained by connecting a protein tag to an N-terminus and/or C-terminus of the protein of any one of (A1)-(A3).

30. The method of claim 29, wherein the nucleic acid molecule capable of expressing the RSB11 protein is any one of the following:
(B1) a DNA molecule shown in SEQ ID NO: 2 or SEQ ID NO: 3;
(B2) a DNA molecule hybridizing with the DNA molecule defined in (B1) under stringent conditions and encoding the RSB11 protein;
(B3) a DNA molecule having 99% or more, 95% or more, 90% or more, 85% or more, or 80% or more identity with any one of the DNA sequences defined in (B1)-(B2) and encoding the RSB11 protein.

31. The method of claim 29, wherein the plant is a monocot or a dicot.

32. The method of claim 31, wherein the monocot is a grass plant.

33. The method of claim 32, wherein the grass plant is a rice plant.

34. The method of claim 33, wherein the rice plant is rice.


SEQUENCE LISTING (25 sequences)

SEQ ID NO: 1 - protein, 820 aa, synthetic construct
MSPLHTHLGFFLLLLLFSLHNPSCSAAANDTLAVGQVLAVGEKLVSRNGKFALGFYKPALPEGIASKYGNITSPGWYLAIWFNKIPVCTTVWVANRERPITDLEIKLTQLKFSQNGSSLAIIINRATEYTVWSRQIANRTAQAKTSMNTSAILLDSGNLVIESIPDVYLWQSFDEPTDLALPGAKFGWNKVTRLHRTGISKKNLIDPGLGPYSVQLNERGIILWRRDPYMEYWTWSSVQLTNMLIPLLNSLLEMNAQTKGFLTPNYTNNNEEEYFMYHSSDESSSSFVSIDMSGQLKLSIWSQANQSWQEVYAQPPDPCTPFATCGPFSVCNGNSDLFCDCMESFSQKSPQDWELKDRTAGCFRNTPLDCPSNKSSTDMFHTITRVALPANPEKIEDATTQSKCAESCLSNCSCNAYAYKDSTCFVWHSELLNVKLHDSIESLSEDTLYLRLAAKDMPATTKNKQKPVVVAVTAASIAGFGLLMLMLFFLIWRNKFKCCGVTLHHNQGNSGIIAFRYTDLSHATKNFSEKLGSGGFGSVFKGVLRDSTTIAVKRLDGSHQGEKQFRAEVSSLGLIQHINLVKLIGFCCEGDKRLLVYEHMVNGSLDAHLFHSNGAVLDWNTRHQIAIGVARGLSYLHESCRECIIHCDIKPENILLEASFAPKIADFGMAAFVGRDFSRVLTTFRGTKGYLAPEWLSGVAITPKVDVYSFGMVLLEIISGRRNLSEAYTSNHYHFDYFPVQAISKLHEGSVQNLLDPELHGDFNLEEAERVCKVACWCIQEDEIDRPTMGEVVRFLEGLQEVDMPPMPRLLAAITERSHI

SEQ ID NO: 2 - DNA, 2463 bp, synthetic construct
  source 1..2463: mol_type = other DNA
atgtctccactccacacccatctcgggttcttcctcctcctcctcctcttctccttgcacaatccatcatgctccgccgcggcgaatgataccctcgcggtggggcaggtgctcgccgtcggggagaagctcgtctcgaggaacggcaagttcgcgctcggcttctacaagccggcactccctgaaggcattgcaagtaagtatggcaacatcacctctcctggatggtaccttgccatatggttcaacaagatcccggtttgtaccaccgtgtgggttgctaatagggagaggccaatcactgatctagagatcaagctaacacagctgaaattctcacaaaatggcagtagccttgccatcatcataaaccgtgctactgaatacacagtatggtccagacaaattgccaataggacagcacaagctaaaaccagcatgaacactagtgcaattctcttggacagtggaaatcttgtcatagaaagcatccctgatgtttatctgtggcagagctttgatgaaccgacggatcttgcgcttcctggcgcaaagtttgggtggaataaggtcaccaggttgcatcgtactggtatatcaaagaaaaaccttattgatccgggtctcggcccatattccgttcaactgaacgagaggggcattatcctgtggcgtcgagatccttacatggagtattggacctggtcatctgtccaattgacaaatatgctcataccattactcaattcgctactagaaatgaatgcacagaccaaaggtttccttaccccaaattatactaacaacaacgaagaggaatacttcatgtaccattcatcagatgaatcctcttcctcatttgtctcaatagacatgtctggccagctcaagttgagcatttggtcacaagctaatcagtcttggcaagaagtatatgcacagcctcctgatccatgcactccatttgcaacctgcggaccttttagcgtatgcaatggcaattcagatttattttgtgattgtatggagagcttctctcagaagtcacctcaagattgggagctcaaggatcgaaccgcaggttgcttcagaaatacgccattagattgccctagtaacaagagctcgacagacatgttccacacaataactcgggtcgcattgccagctaaccccgaaaaaatagaagatgctaccactcaaagcaaatgtgccgaatcttgtctcagtaactgctcctgcaatgcttatgcctacaaagatagcacatgctttgtctggcatagtgaactccttaatgtaaagctacatgacagcattgaaagtctttctgaagatactctttaccttcgcctagctgccaaagatatgccagctacgaccaaaaacaaacaaaaaccagttgttgtggctgttaccgctgcaagcattgccggttttgggttactaatgctcatgctattcttcttgatttggcggaacaaattcaagtgttgtggtgtaacattacaccacaaccaaggcaacagtggaataatagccttcagatacactgatttaagtcatgctaccaaaaatttctcagaaaagttggggtcaggtggttttggttctgtgttcaagggagtattaagagactcaactactattgcggtgaaaaggctcgatggttcacatcagggagaaaagcaattcagggctgaggtgagttcacttggattgattcaacatataaatcttgtcaaattgattggtttctgctgtgaaggtgataagaggttacttgtgtatgaacacatggtaaatggttctcttgatgcccacctttttcatagcaatggtgctgtcctagactggaacactaggcatcaaatagccataggagttgctagaggattgtcctacttgcatgagagttgccgtgaatgcatcatacactgcgatattaagccagaaaatatacttcttgaagcatcatttgctcctaaaattgcagactttgggatggcggcatttgtgggaagggattttagtcgggttctaactactttcaggggaactaaagggtatcttgccccagagtggcttagcggagtcgctattactccaaaagttgatgtttacagttttggtatggtactgctagaaatcatatcaggaagaaggaatttatctgaagcatacacaagcaaccattatcattttgattattttcccgtgcaagccatcagcaaacttcatgagggaagtgtgcagaatttgcttgatccagaattacatggtgatttcaatttggaagaggctgaaagagtttgtaaagttgcatgttggtgcattcaagaagatgaaattgataggccaacaatgggtgaagtggttcgctttcttgagggtctacaggaggttgacatgccccccatgccaagactacttgcagccataaccgaaaggtctcatatatag

SEQ ID NO: 3 - DNA, 2463 bp, synthetic construct
  source 1..2463: mol_type = other DNA
atgtctccactccacacccatctcgggttcttcctcctcctcctcctcttctccttgcacaatccatcatgctccgccgcggcgaatgataccctcgcggtggggcaggtgctcgccgtcggggagaagctcgtctcgaggaacggcaagttcgcgctcggcttctacaagccggcactccctgaaggcattgcaagtaagtatggcaacatcacctctcctggatggtaccttgccatatggttcaacaagatcccggtttgtaccaccgtgtgggttgctaatagggagaggccaatcactgatctagagatcaagctaacacagctgaaattctcacaaaatggcagtagccttgccatcatcataaaccgtgctactgaatacacagtatggtccagacaaattgccaataggacagcacaagctaaaaccagcatgaacactagtgcaattctcttggacagtggaaatcttgtcatagaaagcatccctgatgtttatctgtggcagagctttgatgaaccgacggatcttgcgcttcctggcgcaaagtttgggtggaataaggtcaccaggttgcatcgtactggtatatcaaagaaaaaccttattgatccgggtctcggcccatattccgttcaactgaacgagaggggcattatcctgtggcgtcgagatccttacatggagtattggacctggtcatctgtccaattgacaaatatgctcataccattactcaattcgctactagaaatgaatgcacagaccaaaggtttccttaccccaaattatactaacaacaacgaagaggaatacttcatgtaccattcatcagatgaatcctcttcctcatttgtctcaatagacatgtctggccagctcaagttgagcatttggtcacaagctaatcagtcttggcaagaagtatatgcacagcctcctgatccatgcactccatttgcaacctgcggaccttttagcgtatgcaatggcaattcagatttattttgtgattgtatggagagcttctctcagaagtcacctcaagattgggagctcaaggatcgaaccgcaggttgcttcagaaatacgccattagattgccctagtaacaagagctcgacagacatgttccacacaataactcgggtcgcattgccagctaaccccgaaaaaatagaagatgctaccactcaaagcaaatgtgccgaatcttgtctcagtaactgctcctgcaatgcttatgcctacaaagatagcacatgctttgtctggcatagtgaactccttaatgtaaagctacatgacagcattgaaagtctttctgaagatactctttaccttcgcctagctgccaaagatatgccagctacgaccaaaaacaaacaaaaaccagttgttgtggctgttaccgctgcaagcattgccggttttgggttactaatgctcatgctattcttcttgatttggcggaacaaattcaagtgttgtggtgtaacattacaccacaaccaaggcaacagtggaataatagccttcagatacactgatttaagtcatgctaccaaaaatttctcagaaaagttggggtcaggtggttttggttctgtgttcaagggagtattaagagactcaactactattgcagtgaaaaggctcgatggttcacatcagggagaaaagcaattcagggctgaggtgagttcacttggattgattcaacatataaatcttgtcaaattgattggtttctgctgtgaaggtgataagaggttacttgtgtatgaacacatggtaaatggttctcttgatgcccacctttttcatagcaatggtgctgtcctagactggaacactaggcatcaaatagccataggagttgctagaggattgtcctacttgcatgagagttgccgtgaatgcatcatacactgcgatattaagccagaaaatatacttcttgaagcatcatttgctcctaaaattgcagactttgggatggcggcatttgtgggaagggattttagtcgggttctaactactttcaggggaactaaagggtatcttgccccagagtggcttagcggagtcgctattactccaaaagttgatgtttacagttttggtatggtactgctagaaatcatatcaggaagaaggaatttatctgaagcatacacaagcaaccattatcattttgattattttcccgtgcaagccatcagcaaacttcatgagggaagtgtgcagaatttgcttgatccagaattacatggtgatttcaatttggaagaggctgaaagagtttgtaaagttgcatgttggtgcattcaagaagatgaaattgataggccaacaatgggtgaagtggttcgctttcttgagggtctacaggaggttgacatgccccccatgccaagactacttgcagccataaccgaaaggtctcatatatag

SEQ ID NO: 4 - DNA, 3177 bp, synthetic construct
  source 1..3177: mol_type = other DNA
atgcgtcatgctgtgggtcatgtatgccgtaatgtgccgagttcaggtgctcaagatccataacctcgatcaaatagacattgaagtgtacaagggcatgcctcttgtctcgccacacttgaccaagatagaactctctggtatagagttgaagaattgcttccttaacttctcgagctgtcctgcactgaaggagctatatttcaccaagaattgcggctctgattccgtttacgagattttgtcccggtcgatacaatgtcttcatattttccattgtcaattcggtgagtatcaccgcacagacatttatgcaccgagcctcattacgctcttacttgaaggtttttgtggcagaactccttttctgggaataatgccctcattagtcgaagcatgtcagaccccaccaggattgcgatgactcgtgtagtaatagttactctggtaattgtgaagatgaatactgtgataggtgtcattgtagctatgaggttagcgacgattctgaaagcttgctcctaggaggtctaacagaagctgagaatttgaagttgatagctggacctaacatagtaagcttacgttaacatcttatcagatatctccttaatttatgttatttcctgtatctgattcaccattttaacatcttaagaatatgtttcatgcattactatgtcttggctcttaaattattgtattcacaacaaaatttggttgactttctcgcttgcactttatccagtttattttcataagtgatttaagatggtgccctttgtttagcaagctgaagtgtttgttactcaatgagtggtgtcttgcttctaatttctcggcactagcttgtattcttgaacactcaccagttctacagaagctcactcttaaaatttccaaggtatatgtttttacaactgctgcattgttaaagttgttgtgcatgattaattgaaggtgaaatttatttccctgctacaggaatataaatctatggtggaattggaaacagaagagaatgacaatccattgtggaaaccagctgcaatatcagaacaccttaaggtagtgaaagtccactgtaaagaagttgatgagggggtttacaaaattgggaagtggttgagtacattggacataaaagttattatcaagcagaggaaacaatcaccaaaacgtaagcttactattgacatttttcattcttccagtatttctgaaatttgtattttttttatttttttggcaaatgatagaaagaaataactagaactggaaatacagatttagaaaaaaaaagaaggaaagggaaactgaaaaaagaactgtatttagaagggataaaaggaaaacatgatccattgtgttttgttgtcaagtacgatccatgaaaacatacagagtttttttcctgttgaactgagttataaatagaaacagaatatcttgacttctacaatttgaactgtttacttgttgaatatctttcatttcatgttaatcttgttaacacagtttccgcatgagtaattctggacagtaacacaatttgttgactagtggaatagatcttagtacctgacattgatacattatatggcttcttatttaccttcatctttgaaattattgttccttatctttgaaagggcattcagtattgcacatattgttgatcgaatcgcacatctgtgcctcaacttattgtactttggatactgatgcagtcttctgtctggaggaaggaaatatttgttgatgcctggtcaaaaaaagaaagaagacgcagtgaatcctagtagtggcgcctttttttttcccctaaggtgcttggcgaggattttctcatcgaggtgttactttttccttttcttataacctgtctagtttttactccgacttactatgatacaattggcagttcttctgccggttcagttattttttaaaaaaaaatgagaaagaagtaacaatagattagctagatgaagcacctcttagttactccctccgtttcacaatgtaagtcattctagcatttcccacatttatattgatattaataaatttagataaatatcaatatgaatgtgggaaatgctagaatgacttatattgtgaaacggaggaagtatttagtaatgccaggcttaaagttcgcagtgacaagaactatctgtcttgtctatgagattgctaggtcagtttcgctaggaagaacaactgtgatcccgttcatttctccaataagagttggatgaagatatagattttcatggcacgtttttcaaactgctaaatggtgtgtttcgtgcgaaaactttatatatgaaagttgctttaaaatattatattaatctatttttcaagttcataataattaaaactcaatcaatcatacgttaataccacatcattttaagtaaaaaacttaatctttatcttcatcttcatcttcaaaaaaaaaaaaaaacaccaccggcgttttggaatgagttgtttagcagttaatctgtagctggttttcctgcagtcgtgttcttttcgctaatgtgctaatataagaatataacgcgtagtgctgtcacttgctacttgagattatatatttgttcattaggccggtacaagtggggccatgtgcagtcctgtgtcgtcgtaatcgacaataatctcaagttgccgtgtgcctgctgaagttgaacagtcaaacaatttgtgttctgtttctatgaatccattctcttcttccccgtcaattaaaacagctaacaaagcaacacacatttgtgcaggttcacactaagcttcctgttcattttcttctccaccagttcacaggagtacagcaactgggagctgcagctgcaagcatccaaatcttccagcctggagaggcatgtagctatagctttacttggtctttggtcattgaccaaagtaaactccaatagagactagtctacaactgctccagtcatgtccatatcaacacttaatctgacagttcagtttcccggcatctagtgtcgctgctcaatatcaacagtcaaacttgcatcaattttcctgtatatatcaccacacactcccaagtcccaacaacacctacttctcacacatccatgctgctgcctctccaatccatcctctgctactttctccaacatttcc

SEQ ID NO: 5 - DNA, 27 bp, synthetic construct
  source 1..27: mol_type = other DNA
tatgaggttagcgacgattctgaacgc

SEQ ID NO: 6 - DNA, 20 bp, synthetic construct
  source 1..20: mol_type = other DNA
ggtgaatcagatacaggaaa

SEQ ID NO: 7 - DNA, 154 bp, synthetic construct
  source 1..154: mol_type = other DNA
tatgaggttagcgacgattctgaacgcktgctcctaggaggtctaacagaagctgagaatttgaagttgatagctggacctaacatagtaagcttacgttaacatcttatcagatatctccttaatttatgttatttcctgtatctgattcacc

SEQ ID NO: 8 - DNA, 18 bp, synthetic construct
  source 1..18: mol_type = other DNA
ataccctcgcggtggggc

SEQ ID NO: 9 - DNA, 22 bp, synthetic construct
  source 1..22: mol_type = other DNA
aggctcgatggttcacatcagg

SEQ ID NO: 10 - DNA, 22 bp, synthetic construct
  source 1..22: mol_type = other DNA
aatgccgccatcccaaagtctc

SEQ ID NO: 11 - DNA, 36 bp, synthetic construct
  source 1..36: mol_type = other DNA
ccccctcgaggtcgacgtgggtcatgtatgccgtaa

SEQ ID NO: 12 - DNA, 37 bp, synthetic construct
  source 1..37: mol_type = other DNA
ttggcgtcttccatggagaagtaggtgttgttgggac

SEQ ID NO: 13 - DNA, 40 bp, synthetic construct
  source 1..40: mol_type = other DNA
ccatggtcgatcgacagatctatgcgtcatgctgtgggtc

SEQ ID NO: 14 - DNA, 46 bp, synthetic construct
  source 1..46: mol_type = other DNA
ccgggtaccgagctcgaattctgtgagaagtaggtgttgttgggac

SEQ ID NO: 15 - DNA, 38 bp, synthetic construct
  source 1..38: mol_type = other DNA
tcgagctcggtacccgggatgtctccactccacaccca

SEQ ID NO: 16 - DNA, 39 bp, synthetic construct
  source 1..39: mol_type = other DNA
ggtcgactctagaggatccttagaacccgactaaaatcc

SEQ ID NO: 17 - DNA, 40 bp, synthetic construct
  source 1..40: mol_type = other DNA
tctgcactaggtacctgcagatgtctccactccacaccca

SEQ ID NO: 18 - DNA, 37 bp, synthetic construct
  source 1..37: mol_type = other DNA
atggatccgtcgacctgcaggaacccgactaaaatcc

SEQ ID NO: 19 - DNA, 20 bp, synthetic construct
  source 1..20: mol_type = other DNA
acgcgtagtgctgtcacttg

SEQ ID NO: 20 - DNA, 20 bp, synthetic construct
  source 1..20: mol_type = other DNA
tgatgatggcaaggctactg

SEQ ID NO: 21 - DNA, 23 bp, synthetic construct
  source 1..23: mol_type = other DNA
ctagctagagtcgagaattcagt

SEQ ID NO: 22 - DNA, 25 bp, synthetic construct
  source 1..25: mol_type = other DNA
ataccctcgcggtggggcaggtgct

SEQ ID NO: 23 - DNA, 26 bp, synthetic construct
  source 1..26: mol_type = other DNA
ataccctcgcggtgggggcaggtgct

SEQ ID NO: 24 - DNA, 26 bp, synthetic construct
  source 1..26: mol_type = other DNA
ataccctcgcggtggcggcaggtgct

SEQ ID NO: 25 - DNA, 24 bp, synthetic construct
  source 1..24: mol_type = other DNA
ataccctcgcggtgggcaggtgct